SEQUENCE LISTING (77 sequences)

SEQ ID NO: 1 - DNA, 20 bp, Artificial Sequence
  Synthetic oligonucleotide probe
anacagatttccgtgggacc

SEQ ID NO: 2 - DNA, 20 bp, Artificial Sequence
  Synthetic oligonucleotide probe
tagtccgagctgggcagana

SEQ ID NO: 3 - DNA, 18 bp, Artificial Sequence
  Synthetic oligonucleotide probe
ggcgcggggtcgtggana

SEQ ID NO: 4 - DNA, 21 bp, Artificial Sequence
  Synthetic oligonucleotide probe
anaaaccacctgcactaggga

SEQ ID NO: 5 - DNA, 20 bp, Artificial Sequence
  Synthetic oligonucleotide probe
antccggtgtcgaagtgggg

SEQ ID NO: 6 - DNA, 20 bp, Artificial Sequence
  Synthetic oligonucleotide probe
gagcaaggtggatgcacana

SEQ ID NO: 7 - DNA, 18 bp, Artificial Sequence
  Synthetic oligonucleotide probe
anaccagggggagcatag

SEQ ID NO: 8 - DNA, 20 bp, Artificial Sequence
  Synthetic oligonucleotide probe
tggtggatggtggggctant

SEQ ID NO: 9 - DNA, 19 bp, Artificial Sequence
  Synthetic oligonucleotide probe
ggactggggcctcggaana

SEQ ID NO: 10 - DNA, 21 bp, Artificial Sequence
  Synthetic oligonucleotide probe
gtacctcctatccacgtcana

SEQ ID NO: 11 - DNA, 19 bp, Artificial Sequence
  Synthetic oligonucleotide probe
ctgtgaccagctggacana

SEQ ID NO: 12 - DNA, 17 bp, Artificial Sequence
  Synthetic oligonucleotide probe
ctgagcacnggatgacc

SEQ ID NO: 13 - DNA, 35 bp, Artificial Sequence
  Synthetic oligonucleotide probe
anaggctttcctgacccagctggatgagctgctaa

SEQ ID NO: 14 - DNA, 26 bp, Artificial Sequence
  Synthetic oligonucleotide probe
tgggacccagcccagccccccccana

SEQ ID NO: 15 - DNA, 17 bp, Artificial Sequence
  Synthetic oligonucleotide probe
cacccccargacgcccc

SEQ ID NO: 16 - DNA, 26 bp, Artificial Sequence
  Synthetic oligonucleotide probe
gnaggcgaccccttacccgcatctcc

SEQ ID NO: 17 - DNA, 27 bp, Artificial Sequence
  Synthetic oligonucleotide probe
tttcgccccaacggtctcttggacana

SEQ ID NO: 18 - DNA, 17 bp, Artificial Sequence
  Synthetic oligonucleotide probe
tggagcagngggtgacc

SEQ ID NO: 19 - DNA, 26 bp, Artificial Sequence
  Synthetic oligonucleotide probe
cnacttgggcctgggcaagaagtcgc

SEQ ID NO: 20 - DNA, 36 bp, Artificial Sequence
  Synthetic oligonucleotide probe
gaggaggccgcctgcctttgtgccgccttcgccanc

SEQ ID NO: 21 - DNA, 17 bp, Artificial Sequence
  Synthetic oligonucleotide probe
gatcctacntccggatg

SEQ ID NO: 22 - DNA, 86 bp, Artificial Sequence
  Synthetic oligonucleotide probe
anaacctgcgccatagtggtggctgacctgttctctgccgggatggtgaccacctcgaccacgctggcctggggcctcctgctcat

SEQ ID NO: 23 - DNA, 24 bp, Artificial Sequence
  Synthetic oligonucleotide probe
tgcagcgtgagcccatctgggana

SEQ ID NO: 24 - DNA, 20 bp, Artificial Sequence
  Synthetic oligonucleotide probe
agtcatgtgaactagctana

SEQ ID NO: 25 - DNA, 20 bp, Artificial Sequence
  Synthetic oligonucleotide probe
anagggtcctgacctcatgc

SEQ ID NO: 26 - DNA, 20 bp, Artificial Sequence
  Synthetic oligonucleotide probe
anatggggagccaccataga

SEQ ID NO: 27 - DNA, 20 bp, Artificial Sequence
  Synthetic oligonucleotide probe
anaatatcagcaacattcac

SEQ ID NO: 28 - DNA, 20 bp, Artificial Sequence
  Synthetic oligonucleotide probe
anatacattgcatcatctat

SEQ ID NO: 29 - DNA, 20 bp, Artificial Sequence
  Synthetic oligonucleotide probe
anactcatagcctcttccca

SEQ ID NO: 30 - DNA, 20 bp, Artificial Sequence
  Synthetic oligonucleotide probe
anatagcacagccaataagc

SEQ ID NO: 31 - DNA, 20 bp, Artificial Sequence
  Synthetic oligonucleotide probe
anatagctgatcaaccaact

SEQ ID NO: 32 - DNA, 20 bp, Artificial Sequence
  Synthetic oligonucleotide probe
anatggacagttacaggaaa

SEQ ID NO: 33 - DNA, 20 bp, Artificial Sequence
  Synthetic oligonucleotide probe
anactttctccagcacccaa

SEQ ID NO: 34 - DNA, 20 bp, Artificial Sequence
  Synthetic oligonucleotide probe
anatgggggaaagtggctta

SEQ ID NO: 35 - DNA, 17 bp, Artificial Sequence
  Synthetic oligonucleotide probe
acagggtttcagacana

SEQ ID NO: 36 - DNA, 17 bp, Artificial Sequence
  Synthetic oligonucleotide probe
anacatactttcacaaa

SEQ ID NO: 37 - DNA, 17 bp, Artificial Sequence
  Synthetic oligonucleotide probe
anagactggggtggggg

SEQ ID NO: 38 - DNA, 16 bp, Artificial Sequence
  Synthetic oligonucleotide probe
anagaattttgatgcc

SEQ ID NO: 39 - DNA, 16 bp, Artificial Sequence
  Synthetic oligonucleotide probe
anaggacatggtttaa

SEQ ID NO: 40 - DNA, 16 bp, Artificial Sequence
  Synthetic oligonucleotide probe
anatatcaagtgttgg

SEQ ID NO: 41 - DNA, 16 bp, Artificial Sequence
  Synthetic oligonucleotide probe
anagttatgtaataac

SEQ ID NO: 42 - DNA, 16 bp, Artificial Sequence
  Synthetic oligonucleotide probe
tatctatatctatana

SEQ ID NO: 43 - DNA, 16 bp, Artificial Sequence
  Synthetic oligonucleotide probe
cagggttttagacana

SEQ ID NO: 44 - DNA, 16 bp, Artificial Sequence
  Synthetic oligonucleotide probe
anatgttagaaagttg

SEQ ID NO: 45 - DNA, 16 bp, Artificial Sequence
  Synthetic oligonucleotide probe
gttggttgtgtggang

SEQ ID NO: 46 - DNA, 20 bp, Artificial Sequence
  Synthetic oligonucleotide probe
aacatccatataaagctatc

SEQ ID NO: 47 - DNA, 20 bp, Artificial Sequence
  Synthetic oligonucleotide probe
ctgcctcaccaccgtgctgg

SEQ ID NO: 48 - DNA, 17 bp, Artificial Sequence
  Synthetic oligonucleotide probe
agaaaggcatggagana

SEQ ID NO: 49 - DNA, 16 bp, Artificial Sequence
  Synthetic oligonucleotide probe
anagggatacagttga

SEQ ID NO: 50 - DNA, 16 bp, Artificial Sequence
  Synthetic oligonucleotide probe
anattacattttctat

SEQ ID NO: 51 - DNA, 16 bp, Artificial Sequence
  Synthetic oligonucleotide probe
anatagctgatcaacc

SEQ ID NO: 52 - DNA, 16 bp, Artificial Sequence
  Synthetic oligonucleotide probe
anagagggtatacttt

SEQ ID NO: 53 - DNA, 20 bp, Artificial Sequence
  Synthetic oligonucleotide probe
cctgggctgcaaggtgtaag

SEQ ID NO: 54 - DNA, 20 bp, Artificial Sequence
  Synthetic oligonucleotide probe
ctgcaggatgtccaggaaga

SEQ ID NO: 55 - DNA, 21 bp, Artificial Sequence
  Synthetic oligonucleotide probe
anatcatacagtgcaacgana

SEQ ID NO: 56 - DNA, 15 bp, Artificial Sequence
  Synthetic oligonucleotide probe
ggcagatctccaana

SEQ ID NO: 57 - DNA, 16 bp, Artificial Sequence
  Synthetic oligonucleotide probe
anagccccttcttgga

SEQ ID NO: 58 - DNA, 13 bp, Artificial Sequence
  Synthetic oligonucleotide probe
cttccagataana

SEQ ID NO: 59 - DNA, 20 bp, Artificial Sequence
  Synthetic oligonucleotide probe
ancatgcggtaggtggtggg

SEQ ID NO: 60 - DNA, 20 bp, Artificial Sequence
  Synthetic oligonucleotide probe
tgagatggtggcctcggana

SEQ ID NO: 61 - DNA, 16 bp, Artificial Sequence
  Synthetic oligonucleotide probe
gnaggcgccctcgcca

SEQ ID NO: 62 - DNA, 19 bp, Artificial Sequence
  Synthetic oligonucleotide probe
cagggagaagcttctgana

SEQ ID NO: 63 - DNA, 18 bp, Artificial Sequence
  Synthetic oligonucleotide probe
anagtctacacgagttgg

SEQ ID NO: 64 - DNA, 20 bp, Artificial Sequence
  Synthetic oligonucleotide probe
ttattgatggtcagcggtna

SEQ ID NO: 65 - DNA, 19 bp, Artificial Sequence
  Synthetic oligonucleotide probe
anactcatcatctgcgctt

SEQ ID NO: 66 - DNA, 20 bp, Artificial Sequence
  Synthetic oligonucleotide probe
tggacgatgacattcagana

SEQ ID NO: 67 - DNA, 16 bp, Artificial Sequence
  Synthetic oligonucleotide probe
ttgaactctgcttana

SEQ ID NO: 68 - DNA, 18 bp, Artificial Sequence
  Synthetic oligonucleotide probe
anacgcggtagatgccca

SEQ ID NO: 69 - DNA, 15 bp, Artificial Sequence
  Synthetic oligonucleotide probe
atgtccgtggccana

SEQ ID NO: 70 - DNA, 17 bp, Artificial Sequence
  Synthetic oligonucleotide probe
gnaggctgccttcagtg

SEQ ID NO: 71 - DNA, 20 bp, Artificial Sequence
  Synthetic oligonucleotide probe
anacacrcctacrcraccrc

SEQ ID NO: 72 - DNA, 19 bp, Artificial Sequence
  Synthetic oligonucleotide probe
anaaacaaactaacrcrca

SEQ ID NO: 73 - DNA, 19 bp, Artificial Sequence
  Synthetic oligonucleotide probe
aacraaaatatatacrana

SEQ ID NO: 74 - DNA, 18 bp, Artificial Sequence
  Synthetic oligonucleotide probe
caacraatctaacrcana

SEQ ID NO: 75 - DNA, 18 bp, Artificial Sequence
  Synthetic oligonucleotide probe
taaaacraccrccraana

SEQ ID NO: 76 - DNA, 19 bp, Artificial Sequence
  Synthetic oligonucleotide probe
aacrcrataaaacraacna

SEQ ID NO: 77 - DNA, 30 bp, Artificial Sequence
  Synthetic oligonucleotide probe
anatttttaaaacttaaaatactaaatana

(19) United States
(12) Patent Application Publication
(10) Pub. No.: US 2004/0110200 A1
(43) Pub. Date: Jun. 10, 2004

(54) Polymorphism detection among homologous sequences

(76) Inventors: Risa Peoples, Palo Alto, CA (US); Reuel B. Van Atta, Mountain View, CA (US)

Correspondence Address: Ralph T. Lilore, 3rd Floor, 371 Franklin Avenue, P.O. Box 510, Nutley, NJ, 07110, US

(21) Appl. No.: 10/651,833

(22) Filed: Aug. 29, 2003

Related U.S. Application Data
(60) Provisional application No. 60/407,598, filed on Aug. 29, 2002.

(51) Int. Cl. C12Q 1/68

(52) U.S. Cl. 435/6

(57) ABSTRACT

The present invention is drawn to a flexible oligonucleotide hybridization system for detecting polymorphisms among sequences sharing high sequence homology, utilizing capture and reporter probes which provide for allelic discrimination and selection of target from among the homologous sequences.

### TECHNICAL FIELD

The field of this invention is nucleic acid sequence detection, and more specifically, the detection of single nucleotide polymorphisms (SNPs) and other polymorphisms of interest in genetic regions exhibiting high sequence homology.

### BACKGROUND

The general principle of oligonucleotide hybridization-based SNP detection is that oligonucleotides can be designed to demonstrate significantly more efficient hybridization to “perfect match” target-regions relative to those regions containing a single base-pair mismatch under defined conditions. In order for this discrimination to be reliably attained, oligonucleotide length is limited, such that single nucleotide differences impact potential hybridization complex melt temperatures. Generally, this limits oligonucleotide probes to a maximum of no more than fifty base pairs for the majority of described hybridization conditions. A special dilemma exists for SNP detection from regions sharing high sequence homology, and often identity, around the SNP site with extraneous loci. In this circumstance, use of a single oligonucleotide for assaying a complex sequence mix is confounded by cross-hybridization to these other loci. The Invader™ assay is particularly vulnerable in this area as it necessarily evaluates a very short target region.

Other oligonucleotide hybridization-based SNP detection platforms mitigate these effects by “pre-selecting” the target of interest by prior PCR amplification using locus-specific primer sequences. There are several limitations of this approach. First, only two locus-specific sequences can be used as forward and reverse primers. Therefore, undefined polymorphisms under these sites will severely impact specificity and can lead to asymmetric allele amplification. Often, in an attempt to achieve reliable primer specificity, sequence far from SNP sites must be used, necessitating the generation of very long PCR products of up to tens of kilobases. This requires meticulous template preparation and, even in experienced hands, is often unreliable. The utility of PCR in the clinical diagnostics laboratory is further limited by its intrinsic geometric amplification process which makes quantitation difficult and the potential for errors due to amplicon contamination.

Unfortunately, the circumstance of highly homologous sequences existing with the potential to complicate clinically relevant SNP detection is not at all uncommon. Mechanisms contributing to this situation include gene duplication, copying of processed transcripts back into DNA (“pseudogene formation”), gene evolution by exonic shuffling, and duplication of large blocks of DNA (usually on the order of hundreds of kilobases).

The cytochrome P450 genes whose protein products are responsible for the inactivation and degradation of the majority of drugs are examples of gene duplications. These loci have emerged through gene copying and subsequent divergent natural selection. The genes share strong homology with each other and usually with non-functioning pseudogenes as well. Other examples include the major histocompatibility complex genes important in pre-transplantation diagnostics, and the globin genes, important in the hemoglobinopathies such as the thallasemias. Cross-hybridization with homologous sequences confounds standard hybridization and PCR-based methodologies. To date, high-throughput and cost-effective methods for assaying these loci have not been produced.

The duplication of blocks of DNA over hundreds of kilobases is a subject of particular interest due to the potential for these blocks to misalign and lead to further duplication or deletion events, often with clinically important consequences. Such duplications are referred to as paralogues and can demonstrate particularly high homology among themselves, often on the order of >99%. Paralogous regions are extremely problematic for diagnostics, as “locus-defining” nucleotides allowing the discrimination of two paralogous regions are often themselves subject to mutation events substituting the paralogous sequence and thus, conferring regional identity with the paralogue. That is, a mutation often results from the replacement of a single nucleotide over a short region with the nucleotide of the paralogue, making the two sites now indistinguishable.

That this occurs so many times at so many sites is not entirely an accident. Many SNP mutations arise not by simple mismatch errors, but by gene conversion mutations. In areas that share high homology with other sequences, endogenous scanning and repair mechanisms often mistakenly identify a locus-specific base pair as an error and will use the sequence of the paralogous locus as a template for excision of the correct nucleotide and substitution with the sequence from the paralogue. In many cases where genes of clinical relevance reside within a paralogue, locus-defining nucleotides have been identified that can be used in molecular diagnostics for locus-specificity. Examples include the gene SMN 1, implicated in spinal muscular atrophy and the gene NCF1, important in many cases of chronic granulomatous disease. In both cases, nucleotide substitutions leading to an inactive gene product occur through presumed conversion mutations. In each case, the currently-available assays allowing concurrent evaluation of the mutation site with the presence of well-characterized locus-defining nucleotides are cumbersome and expensive.

What is needed, therefore, are improved assays for detecting SNPs within regions of high sequence homology. Such a platform must be capable of identifying specific mutations or polymorphisms in conjunction with site-defining nucleotides. Ideally, such an assay would also provide improvements in target sensitivity and platform flexibility for evaluation of different mechanisms of mutations.

Relevant Literature

Articles that describe various techniques for detecting deletions and duplications include: Yau et al., J. Med. Genet. 1996; 33(7):550-558; Bentz et al., Genes Chromosomes Cancer 1998; 21(2):172-175; Geschwind et al., Dev. Genet. 1998; 23(3):215-229; Armour et al., Nucleic Acids Res. 2000; 28(2):605-609; Lindblad-Toh et al., Nat. Biotechnol. 2000; 18(9):1001-1005; Ruiz-Ponte et al., Clin. Chem. 2000; 46(10):1574-1582; Jung et al., Clin. Chem. Lab. Med 2000; 38(9):833-836; Kariyazono et al., Mol. Cell. Probes 2001; 15(2):71-73; Antonarakis, Nat. Genet. 2001; 27(3):230-232; Hodgson et al., Nat. Genet. 2001; 29(4):459-464.

Nucleic acid crosslinking probes for DNA/RNA diagnostics are disclosed in Wood et al., Clin. Chem. 1996; 42(S6):S196. Crosslinker-containing probes have been reported to be able to discriminate between single-base polymorphic sites in target sequences in solution-based hybridization assays. Zehnder et al., Clin. Chem. 1997; 43(9):1703-1708.

### SUMMARY OF THE INVENTION

In accordance with the objects outlined above, the present invention provides improved methods for genotyping a target nucleic acid sequence in a sample, where the sample comprises the target sequence of interest and one or more extraneous sequences having high sequence homology to the target sequence. In the preferred embodiment, the target nucleic acid sequence comprises an interrogation region and a locus-specific region, and the method comprises the steps of: adding at least one capture probe and at least one reporter probe to the sample, wherein the capture probe comprises a sequence substantially complementary to the interrogation region of the target sequence and the reporter probe comprises a sequence substantially complementary to the locus-specific region of the target sequence. Next, the capture probe is captured and the reporter probe is detected to determine the genotype of the target sequence, and to discriminate between the target sequence and any extraneous sequences sharing high homology to the target sequence that may be present in the sample.

### DETAILED DESCRIPTION OF THE INVENTION

The present invention provides methods for detecting SNPs and other polymorphisms of interest in a locus-specific manner among genetic regions exhibiting high sequence homology, such as paralogous genes. As described herein, the subject methods generally involve adding one or more distinct capture and reporter probes to a sample comprising a target sequence of interest, with the capture probe(s) providing allele specificity and the reporter probe(s) providing locus specificity. The capture and reporter probe system of the present invention allows for the accurate genotyping of a desired target sequence at a target locus while discriminating against similar or identical polymorphisms that may be present in regions of high homology at a different locus, such as a paralogous locus. As used herein, “high sequence homology” refers to homologous sequences having greater than about 70%, more preferably greater than about 80%, most preferably greater than about 85 or 90%, and generally from about 75-99.9% homology.

As is well known in the art, a “paralogous” locus or gene is one which originated by gene duplication and then diverged from the parent copy by mutation and selection or drift. Genetic errors developed in the paralogous sequence can be incorporated back into the parent gene through gene conversion mechanisms and result in inactivation of the original coding sequence, resulting in variable drug responsiveness or phenotypes associated with various diseases.

As noted above, assaying for the presence of these polymorphisms in the parent coding sequence is difficult due to the high sequence homology between the parent and the paralogue(s). The present invention addresses and solves this persistent problem in the art. Capture probes are provided for genotyping a particular polymorphism of interest, while target specificity is conferred by reporter probes recognizing locus-defining nucleotides present only in the target sequence. By separating the capture and reporter functions, cross-reactivity with homologous sequences such as paralogues exhibiting high sequence homology to the target is controlled.

In one embodiment, the present invention provides one or more reporter probes comprising sequences complementary to a locus-specific region in a target sequence. Preferably, the locus-specific region comprises one or more locus-defining nucleotides which are unique to the target sequence and therefore will preferentially hybridize with the reporter probes to the exclusion of homologous sequences lacking such nucleotides. In this manner, locus specificity to the target locus of interest is achieved.

In a preferred embodiment, the invention further provides one or more capture probes having sequences complementary to the target sequence so as to detect a particular polymorphism (e.g., SNP) of interest, as described in more detail herein. The polymorphism may be either inherited or spontaneous, germline or somatic, or a marker of interspecies variation. Polymorphisms or mutations of interest include SNPs as well as substitutions, insertions, translocations, rearrangements, variable number of tandem repeats, short tandem repeats, retrotransposons such as Alu and long interspersed nuclear elements, and the like. Additionally, as described herein, one may also assay for gene dosage abnormalities such as deletions or duplications in parallel with SNP detection. By convention, sequence variants present at frequencies less than 1% are generally considered mutations, whereas those present at higher frequencies are considered polymorphisms. As used herein, the term “polymorphism” means any DNA sequence variation of any type or frequency.

Generally, the method comprises combining one or more reporter probes and one or more capture probes with a sample comprising a target sequence suspected of having a polymorphism of interest. The target sequence may be present as a major component of the DNA from the target or as one member of a complex mixture. The target sequence comprises a locus-specific region to distinguish over regions of high sequence homology (e.g., paralogues) that may also be present in the sample, and may further comprise an interrogation region, a dosage region and/or a control region as described herein. The capture and reporter probes are characterized by having known sequences derived from the gene or genes of interest, with complementarity to the interrogation position and locus-specific regions, respectively, as explained herein. In a further embodiment, additional probe sets directed to other polymorphic sequences of interest and/or a diploid control locus are also provided.

In a preferred embodiment, the capture and reporter probes further comprise first and second detectable labels, respectively. In one embodiment, the first detectable label of the capture probe comprises a molecule that can be captured on a solid support, e.g., biotin, whereas the second detectable label of the reporter probe preferably comprises a reporter molecule, e.g., a fluorophore, an antigen, or other binding-pair partner useful for direct or indirect detection methods. In a particularly preferred-embodiment, the first detectable label allows for separation of the capture probe-target complexes, such as, e.g., a biotinylated probe exposed to streptavidin-coated beads, whereas the second detectable label provides for quantification of signal strength, such as, e.g., fluorescein. The capture probe is then captured and the reporter probe is detected to determine the presence or absence of the polymorphism of interest in the target sequence. In an alternative embodiment, the first detectable label of the capture probe comprises a reporter molecule and the second detectable label of the reporter probe comprises a molecule that can be captured on a solid support.

In an alternative embodiment, an additional polymorphism relating to gene dosage abnormalities is detected following the methods of the present invention. As used herein, gene dosage refers to the quantitative determination of gene copy number present in an individual's genome. Because the normal human genome is diploid, the normal gene dosage for non X-linked genes is two. Whole gene and larger (microscopic and submicroscopic subchromosomal) deletions and duplications (gene dosage of one and three or more, respectively) confer specific phenotypes, and their diagnosis can be of critical clinical importance. As described herein, the present invention also provides methods and compositions for rapidly and accurately determining the gene copy number of genomic regions subject to these types of duplication and/or deletion events, referred to generally herein as “dosage regions.”

Preferably, in this embodiment the sample further comprises a diploid control locus, termed a “diploid region,” and the gene copy number is determined from the ratio of a dosage signal generated by a probe set directed to the dosage region and a diploid signal generated by a probe set directed to the diploid region, as described further herein. Additional probe sets directed to other polymorphisms or mutations in the gene or genes of interest may also be employed concurrently in the same platform for the same clinical sample, providing a complete genetic profile of a given locus.

As will be appreciated by those in the art, the sample may comprise any number of things, including, but not limited to, bodily fluids (including, but not limited to, blood, urine, serum, lymph, saliva, anal and vaginal secretions, perspiration, and semen, of virtually any organism, with mammalian samples being preferred and human samples being particularly preferred); research samples; purified samples, such as purified genomic DNA, RNA, etc.; raw samples, such as bacteria, virus, genomic DNA, mRNA, etc. The sample may comprise individual cells, including primary cells (including bacteria), and cell lines, including, but not limited to, tumor cells of all types (particularly melanoma, myeloid leukemia, carcinomas of the lung, breast, ovaries, colon, kidney, prostate, pancreas and testes), cardiomyocytes, endothelial cells, epithelial cells, lymphocytes (T-cell and B cell), mast cells, eosinophils, vascular intimal cells, hepatocytes, leukocytes including mononuclear leukocytes, stem cells such as haemopoetic, neural, skin, lung, kidney, liver and myocyte stem cells, osteoclasts, chondrocytes and other connective tissue cells, keratinocytes, melanocytes, liver cells, kidney cells, and adipocytes. Suitable cells also include known research cells, including, but not limited to, Jurkat T cells, NIH3T3 cells, CHO, Cos, 923, HeLa, WI-38, Weri-1, MG-63, etc. See the ATCC cell line catalog, hereby expressly incorporated by reference. As will be appreciated by those in the art, virtually any experimental manipulation may have been done on the sample.

By “nucleic acid” or “oligonucleotide” or grammatical equivalents herein means at least two nucleotides covalently linked together. As will be appreciated by those skilled in the art, various modifications of the sugar-phosphate backbone may be done to facilitate the addition of labels, or to increase the stability and half-life of such molecules in physiological environments. The nucleic acids may be single-stranded or double-stranded, as specified, or contain portions of both double-stranded or single-stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguaninc, etc. As used herein, the term “nucleotide” includes nucleotides as well as nucleoside and nucleotide analogs, and modified nucleosides such as labeled nucleosides. In addition, “nucleotide” includes non-naturally occurring analog structures. Thus, for example, the individual units of a peptide nucleic acid (PNA), each containing a base, are referred to herein as a nucleotide. The term “nucleotide” also encompasses locked nucleic acids (LNA). BVraasch and Corey, Chem. Biol. 2001; 8(1): 1-7. Similarly, the term “nucleotide” (sometimes abbreviated herein as “NTP”), includes both ribonucleic acid and deoxyribonucleic acid (sometimes abbreviated herein as “dNTP”).

The terms “target sequence” or “target nucleic acid” or grammatical equivalents herein mean a nucleic acid sequence. In a preferred embodiment, the “target sequence” comprises a locus-specific region as well as an interrogation region suspected of including a polymorphism of interest. In another embodiment, the target sequence further comprises an additional polymorphism of interest, e.g., a deletion or duplication (termed a “dosage region”). Alternatively, the sample may comprise a plurality of distinct target sequences, each having one or more locus-specific regions of interest. By “plurality” as used herein is meant at least two.

The target nucleic acid may come from any source, either prokaryotic or eukaryotic, usually eukaryotic. The source may be the genome of the host, plasmid DNA, viral DNA, where the virus may be naturally occurring or serving as a vector for DNA from a different source, a PCR amplification product, or the like. The target DNA may be a particular allele of a mammalian host, an MHC allele, a sequence coding for an enzyme isoform, a particular gene or strain of a unicellular organism, or the like. The target sequence may be a portion of a gene, a regulatory sequence, genomic DNA, cDNA, RNA including mRNA and rRNA, or others. As is outlined herein, the target sequence may be a target sequence from a sample, or a secondary target such as a product of a genotyping or amplification reaction such as a ligated circularized probe, an amplicon from an amplification reaction such as PCR, etc. Thus, for example, a target sequence from a sample is amplified to produce a secondary target (amplicon) that is detected. Alternatively, what may be amplified is the probe sequence, although this is not generally preferred. Thus, as will be appreciated by those in the art, the complementary target sequence may take many forms. For example, it may be contained within a larger nucleic acid sequence, i.e. all or part of a gene or mRNA, a restriction fragment of a cloning vector or genomic DNA, among others. As is outlined more fully below, probes are made to hybridize to target and/or control sequences to determine the presence, sequence and/or quantity of a target sequence in a sample. Generally speaking, the term “target sequence” will be understood by those skilled in the art.

If required, the target sequence is prepared using known techniques. For example, the sample may be treated to lyse the cells, using known lysis buffers, sonication, electroporation, etc., with purification and amplification occurring as needed, as will be appreciated by those in the art. The sample may be a cellular lysate, isolated episomal element, e.g., YAC, plasmid, etc., virus, purified chromosomal fragments, cDNA generated by reverse transcriptase, amplification product, mRNA, etc. Depending upon the source, the nucleic acid may be freed of cellular debris, proteins, DNA (if RNA is of interest), RNA (if DNA is of interest), size selected, gel electrophoresed, restriction enzyme digested, sheared, fragmented by alkaline hydrolysis, or the like. Importantly, however, and unlike the prior art, the benefits of improved sensitivity and reproducibility may be obtained following the methods of the present invention even without such additional DNA purification steps.

The target sequence may be of any length, with the understanding that longer sequences are more specific. In one embodiment, the target nucleic acid is provided with an average size in the range of about 0.25 to 3 kb. Nucleic acids of the desired length can be achieved, particularly with DNA, by restriction enzyme digestion, use of PCR and primers, boiling of high molecular weight DNA for a prescribed time, and the like. Desirably, at least about 80 mol %, usually at least about 90 mol % of the target sequence, will have the same size. For restriction enzyme digestion, a frequently cutting enzyme may be employed, usually an enzyme with a four-base recognition sequence, or combination of restriction enzymes may be employed, where the DNA will be subject to complete digestion.

Preferably, double-stranded nucleic acids are denatured to render them single-stranded, so as to permit hybridization of the capture and reporter probes of the invention. A preferred embodiment utilizes a thermal step, generally by raising the temperature of the reaction to about 95 degrees C. in an alkaline environment, although chemical denaturation techniques may also be used. Where chemical denaturation has occurred, normally the medium will then be neutralized to permit hybridization. Various media can be employed for neutralization, particularly using mild acids and buffers, such as acetic acid, citric acid, etc. The particular neutralization buffer employed is selected to provide the desired stringency for hybridization to occur during the subsequent incubation.

The reactions outlined herein may be accomplished in a variety of ways, as will be appreciated by those in the art. Components of the reaction may be added simultaneously, or sequentially, in any order, with preferred embodiments outlined below. In addition, the reaction may include a variety of other reagents that may be included in the assays. These reagents include salts, buffers, neutral proteins, e.g., albumin, detergents, etc., that may be used to facilitate optimal hybridization and detection, and/or reduce non-specific interactions. Also reagents that otherwise improve the efficacy of the assay, such as protease inhibitors, nuclease inhibitors, anti-microbial agents, etc., may be used, depending on the sample preparation methods and purity of the target.

The method comprises the steps of denaturing the sample containing the target sequence and then adding at least one capture probe and at least one reporter probe. The target sequence comprises an interrogation region comprising an interrogation position, which is substantially complementary to the at least one capture probe, and a locus-specific region, which is substantially complementary to the at least one reporter probe. The capture probe(s) are then captured and the presence of the reporter probe(s) detected in the captured complex. The presence or absence of a signal from the reporter probe(s) will indicate the presence or absence of the polymorphism of interest in the target sequence from among other genes or regions of high sequence homology in the sample such as paralogous genes.

In a further embodiment, the above method further comprises detecting gene dosage, wherein the target sequence further comprises at least a portion of a genomic sequence that is known to be subject to deletion or duplication events, generally referred to herein as the “dosage region.” The dosage region will generally comprise a plurality of nucleotides, and more preferably, a plurality of contiguous nucleotides. As used herein, the corresponding region in the probe sequence that hybridizes with the dosage region or other sequence of interest is termed the “detection region.” Probes designed to hybridize with a dosage region in a target sequence are also generally referred to herein as “dosage probes.”

In the preferred embodiment, the method comprises the detection of a polymorphism suspected of being present in the target sequence of interest, such as, e.g., a genotyping reaction. As is more fully outlined below, an interrogation region having a position for which sequence information is desired, generally referred to herein as the “interrogation position,” may be detected using at least one capture probe complementary to portions of the interrogation region as described herein. In one embodiment, the interrogation position is a single nucleotide, although in some embodiments, it may comprise a plurality of nucleotides, either contiguous with each other or separated by one or more nucleotides within the interrogation region. As used herein, the corresponding probe base that basepairs with the interrogation position base in a hybridization complex is termed the “detection position.” In the case where the detection position is a single nucleotide, the NTP in the probe that has perfect complementarity to the detection position is called a “detection NTP.”

“Mismatch” is a relative term and meant to indicate a difference in the identity of a base at a particular position, termed the “interrogation position” herein, between two sequences. In general, sequences that differ from wild type sequences are referred to as mismatches. However, particularly in the case of SNPs, what constitutes “wild type” may be difficult to determine as multiple alleles can be observed relatively frequently in the population, and thus “mismatch” in this context requires the artificial adoption of one sequence as a standard. Thus, for the purposes of this invention, sequences are referred to herein as “perfect match” and “mismatch.” “Mismatches” are also sometimes referred to as “allelic variants.” The term “allele,” which is used interchangeably herein with “allelic variant” refers to alternative forms of a gene or portions thereof. Alleles generally occupy the same position on homologous chromosomes. When a subject has two identical alleles of a gene, the subject is said to be homozygous for the gene or allele. When a subject has two different alleles of a gene, the subject is said to be heterozygous for the gene. Alleles of a specific gene can differ from each other in a single nucleotide, or several nucleotides, and can include substitutions, deletions, and insertions of nucleotides. An allele of a gene can also be a form of a gene containing a mutation. The term “allelic variant of a polymorphic region of a gene” refers to a region of a gene having one of several nucleotide sequences among individuals of the same species.

The present invention provides both capture and reporter probes that hybridize to regions of interest within a target sequence or a plurality of target sequences as described herein. In general, probes of the present invention are designed to be complementary to interrogation regions and locus-specific regions of target sequence(s) (either the target sequence of the sample or to other probe sequences) and/or to dosage regions, such that hybridization occurs between the target and the probes of the present invention. This complementarity need not be perfect; there may be any number of base-pair mismatches that will interfere with hybridization between the target sequence and the corresponding detection regions in the probes of the present invention. However, if the number of mutations is so great that no hybridization can occur under even the least stringent of hybridization conditions, the sequence is not a complementary target sequence. Thus, by “substantially complementary” herein is meant that the probe sequences are sufficiently complementary to the corresponding region of the target sequence (e.g. interrogation region, locus-specific region, dosage region, or diploid region) to hybridize under the selected reaction conditions.

Hybridization generally depends on the ability of denatured DNA to anneal when complementary strands are present in an environment below their melting temperature. The higher the degree of desired complementarity between the probe sequence and the region of interest, the higher the relative temperature that can be used. As a result, it follows that higher relative temperatures would tend to make the reaction conditions more stringent, whereas lower temperatures less so. For additional details and explanation of stringency of hybridization reactions, see Current Protocols in Molecular Biology, Ausubel et al. (Eds.).

Generally, the length of the probe and its GC content will determine the thermal melting point (Tm) of the hybrid, and thus the hybridization conditions necessary for obtaining specific hybridization of the probe to the region of interest. These factors are well known to a person of skill in the art, and can also be tested experimentally. The Tm is the temperature (under defined ionic strength and pH) at which 50% of the target sequence hybridizes to a probe. An extensive guide to the hybridization of nucleic acids is found in Tijssen, Hybridization with Nucleic Acid Probes: Theory and Nucleic Acid Probes, Vol. 1, 1993. Generally, stringent conditions are selected to be about 5 C. lower than the Tm for the specific sequence at a defined ionic strength and pH. Highly stringent conditions are selected to be greater than or equal to the Tm point for a particular probe.

Sometimes the term “dissociation temperature” (“Td”) is used to define the temperature at which half of the probe is dissociated from a target nucleic acid. In any case, a variety of techniques for estimating the Tm or Td are available, and generally described in Tijssen, supra. Typically, G-C base pairs in a duplex are estimated to contribute about 3 C. to the Tm, whereas A-T base pairs are estimated to contribute about 2 C., up to a theoretical maximum of about 80-100 C. However, more sophisticated models of Tm and Td are available and appropriate in which G-C stacking interactions, solvent effects, and the like are taken into account. For example, probes can be designed to have a desired dissociation temperature by using the formula: Td (((((3×#GC)+(2×#AT))×37)−562)/#bp)-5; where #GC, #AT, and #bp are the number of guanine-cytosine base pairs, the number of adenine-thymine base pairs, and the number of total base pairs, respectively, involved in the annealing of the probe to the template DNA.

The stability difference between a perfectly matched duplex and a mismatched duplex, particularly if the mismatch is only a single base, can be quite small, corresponding to a difference in Tm between the two of as little as 0.5 C. Tibanyenda et al., Eur. J. Biochem. 1984; 139(1):19-27 and Ebel et al., Biochemistry 1992; 31(48):12083-1286. More importantly, it is understood that as the length of the complementary region increases, the effect of a single base mismatch on overall duplex stability decreases. Thus, where there is a likelihood of mismatches between the probe sequence and the target sequence, it may be advisable to include a longer complementary region in the probe. Alternatively, where one is probing a known interrogation position with a plurality of allele-specific detection probes, it may be advisable to include a shorter complementary region in the probes to improve discrimination.

Thus, the specificity and selectivity of the probe can be adjusted by choosing proper lengths for the complementary regions and appropriate hybridization conditions. When the sample is genomic DNA, e.g., mammalian genomic DNA, the selectivity of the probe sequences must be high enough to identify the correct sequence in order to allow processing directly from genomic DNA. However, in situations in which a portion of the genomic DNA is first isolated from the rest of the DNA, e.g., by separating one or more chromosomes from the rest of the chromosomes, the selectivity or specificity of the probe may become less important.

The length of the probe, and therefore the hybridization conditions, will also depend on whether a single probe is hybridized to the target sequence, or several probes. In a preferred embodiment, several probes are used and all the probes are hybridized simultaneously to the target sequence. With this embodiment, it is desirable to design the probe sequences such that their Tm or Td is similar, such that all the probes will hybridize specifically to the target sequence. These conditions can be determined by a person of skill in the art, by taking into consideration the factors discussed above.

A variety of hybridization conditions may be used in the present invention, including high-, moderate- and low-stringency conditions; see, e.g., Sambrook et al., Molecular Cloning: A Laboratory Manual, 2^{nd} ed., 1989, and Short Protocols in Molecular Biology, Ausubel et al (Eds.), 1992, hereby incorporated by reference. Stringent conditions are sequence-dependent, and will differ depending on specific circumstances. Longer sequences hybridize more specifically at higher temperatures. Stringent conditions will be those in which the salt concentration is less than about 1.0 M sodium ion, typically about 0.01 to 1.0 M sodium ion concentration (or other salts) at pH 7.0 to 8.3, and the temperature is at least about 30° C. for short probes (e.g., 10 to 50 nucleotides) and at least about 60° C. for long probes (e.g., greater than 50 nucleotides) in an entirely aqueous hybridization medium. Stringent conditions may also be achieved with the addition of helix destabilizing agents such as formamide. The hybridization conditions may also vary when a non-ionic backbone, e.g., PNA is used, as is known in the art.

Thus, the assays are generally run under stringency conditions that allow formation of the hybridization complex only in the presence of target. Stringency can be controlled by altering a step parameter that is a thermodynamic variable, including, but not limited to, temperature, formamide concentration, salt concentration, chaotrope salt concentration, pH, organic solvent concentration, etc. These parameters may also be used to control non-specific binding, as is generally outlined in U.S. Pat. No. 5,681,697. Thus it may be desirable to perform certain steps at higher stringency conditions to reduce non-specific binding, as described herein. The skilled artisan will recognize how to adjust the temperature, ionic strength, etc. as necessary to accommodate factors such as probe length and the like.

As will be appreciated by those in the art, the capture and reporter probes of the invention can take on a variety of configurations. The desired probe will have a sequence of at least about 10, more usually at least about 15, preferably at least about 16 or 17 and usually not more than about 1 kilobases (kb), more usually not more than about 0.5 kb, preferably in the range of about 18 to 200 nucleotides (nt), and frequently not more than 50 nt, where the probe sequence is substantially complementary to the above-noted regions of the target sequence.

In the preferred embodiment, one or more reporter probes are provided having sequences substantially complementary to a locus-specific region in the target sequence of interest, and one or more capture probes are provided to detect a polymorphisms suspected of being present in the target sequence such as, e.g. a known SNP or other polymorphism. In this embodiment, the one or more allele-specific capture probes comprise sequences substantially complementary to the interrogation region upstream and downstream of an interrogation position for which sequence information is desired, but differ in the corresponding interrogation NTPs. In this embodiment, the capture probe sequences are substantially complementary to the sequence surrounding the SNP at the interrogation position, but differ at the corresponding interrogation position with respect to the mutant and wild-type sequences, thereby enabling discrimination between normal and mutant genotypes, as described herein.

In another embodiment, particularly suited for gene dosage determinations as described herein, the sequences of a second set of capture and/or reporter probes are selected so as to be substantially complementary to at least a portion of a known deletion or duplication region (termed a “dosage region”) in a gene or genes of interest. In this manner, the dosage region of interest in a given sample may be assayed for and quantified by comparing the resulting dosage signal against a diploid signal obtained from a known diploid locus in the sample, referred to herein as the “diploid region,” using a second set of probes substantially complementary to the diploid region.

Preferably, the diploid region is selected from a relatively unique region of the genome demonstrating minimal homology with other DNA, thereby minimizing the potential for cross-hybridizing sequence affecting signal strength. Sequence homology is easily ascertained through screening of the human genome through the sequence database maintained by the National Center for Biotechnology Information. As one of skill in the art is well aware, sequence from the non-pseudoautosomal X and Y chromosomal regions should be excluded as dosage varies with gender. Additionally, evidence for potential cell toxicity from over- or under-representation of gene dosage can also be inferred by an examination of chromosomal aberrations in cancer cells (Mitelman Database of Chromosome Aberrations in Cancer (2001). Mitelman F, Johansson B and Mertens F (Eds.), http://cgap.nci.nih.gov/Chromosomes/Mitelman). That is, cancer cells, having lost the normal controls over proliferation and DNA repair and being thus subject to the accumulation of mitotic errors, can indicate specific loci that are more likely to be cell-lethal when present in abnormal copy number. The scarcity of either deletions or duplications of a specific locus in tumor specimens can therefore be taken as evidence that the locus is toxic to cells in abnormal dose and, therefore, will be reliably present in diploid copy number in the vast majority of human cells.

Selection of a diploid region in this manner is particularly suited to the development of assays for somatic dosage abnormalities in mixed-cell populations such as human tissues. Alternatively, so-called “housekeeping genes” can be selected as diploid controls. One of skill in the art will recognize these genes as ones that have been identified as requisite for normal cell growth due to the provision by their product of an essential cell function. Because these genes are also unlikely to be present in other than diploid copy number, they also represent good candidates for diploid loci.

A number of different capture and reporter probes, as described in the examples below, can be included in the same probe mixture. For example, two or more reporter probes may be used directed to different portions of the same locus-specific region of the target or to different locus-specific regions within the target sequence of interest, with each probe having distinct probe complementary sequences. With this embodiment one may guard against the possibility of unknown or rare, undefined SNPs significantly altering the efficacy of the assay.

The probe complementary sequence that binds to the target will usually be naturally occurring nucleotides, but in some instances the sugar-phosphate chain may be modified, by using unnatural sugars, by substituting oxygens of the phosphate with sulfur, carbon, nitrogen, or the like, by modification of the bases, or absence of a base, or other modification that can provide for synthetic advantages, stability under the conditions of the assay, resistance to enzymatic degradation, etc. In one embodiment, modified nucleotides are incorporated into the probes that do not affect the Tms.

The probes may further comprise one or more labels (including ligand), such as a radiolabel, fluorophore, chemilumiphore, fluorogenic substrate, chemilumigenic substrate, biotin, antigen, enzyme, photocatalyst, redox catalyst, electroactive moiety, a member of a specific binding pair, or the like, that allows for capture or detection of the crosslinked probe. The label may be bonded to any convenient nucleotide in the probe chain, where it does not interfere with the hybridization between the probe and the target sequence. Labels will generally be small, usually from about 100 to 1,000 Da. The labels may be any detectable entity, where the label may be able to be detected directly, or by binding to a receptor, which in turn is labeled with a molecule that is readily detectable. Molecules that provide for detection in electrophoresis include radiolabels, e.g., ³²P, ³⁵S, etc. fluorescers, such as rhodamine, fluorescein, etc., ligand for receptors and antibodies, such as biotin for streptavidin, digoxigenin for anti-digoxigenin, etc., chemiluminescers, and the like. Alternatively, the label may be capable of providing a covalent attachment to a solid support such as bead, plate, slide, or column of glass, ceramic or plastic.

Preferred labels in the present invention include spectral labels such as fluorescent dyes (e.g., fluorescein isothiocyanate, Texas red, rhodamine, dixogenin, biotin, and the like), radiolabels (e.g., ³H, ¹²⁵I, ³⁵S, ¹⁴c, ³²P, ³³P, etc.), enzymes (e.g., horse-radish peroxidase, alkaline phosphatase, etc.), spectral calorimetric labels such as colloidal gold or colored glass or plastic (e.g. polystyrene, polypropylene, latex, etc.) beads. Enzymes of interest as labels will primarily be hydrolases, particularly phosphatases, esterases and glycosidases, or oxidoreductases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. Thus, a wide variety of labels may be used, with the choice of label depending on sensitivity required, ease of conjugation with the compound, stability requirements, available instrumentation, and disposal provisions.

The label may be coupled directly or indirectly to the molecule to be detected according to methods well known in the art. Non-radioactive labels are often attached by indirect means. Generally, a ligand molecule (e.g., biotin) is covalently bound to a nucleic acid such as a probe, primer amplicon, YAC, BAC or the like. The ligand then binds to an anti-ligand (e.g., streptavidin) molecule which is either inherently detectable or covalently bound to a signal system, such as a detectable enzyme, a fluorescent compound, or a chemiluminescent compound. A number of ligands and anti-ligands can be used. Where a ligand has a natural anti-ligand, for example, biotin, thyroxine, and cortisol, it can be used in conjunction with labeled, anti-ligands. Alternatively, any haptenic or antigenic compound can be used in combination with an antibody. Labels can also be conjugated directly to signal generating compounds, e.g., by conjugation with an enzyme or fluorophore or chromophore.

Means of detecting labels are well known to those of skill in the art. Thus, for example, where the label is a radioactive label, means for detection include a scintillation counter or photographic film as in autoradiography. Where the label is optically detectable, typical detectors include microscopes, cameras, phototubes and photodiodes and many other detection systems which are widely available. In general, a detector which monitors a probe-target nucleic acid hybridization is adapted to the particular label which is used. Typical detectors include spectrophotometers, phototubes and photodiodes, microscopes, scintillation counters, cameras, film and the like, as well as combinations thereof. Examples of suitable detectors are widely available from a variety of commercial sources known to persons of skill. Commonly, an optical image of a substrate comprising a nucleic acid array with particular set of probes bound to the array is digitized for subsequent computer analysis.

Fluorescent labels are preferred labels, having the advantage of requiring fewer precautions in handling, and being amendable to high-throughput visualization techniques. Preferred labels are typically characterized by one or more of the following: high sensitivity, high stability, low background, low environmental sensitivity and high specificity in labeling. Fluorescent moieties, which are incorporated into the labels of the invention, are generally known, including Texas red, dixogenin, biotin, 1- and 2-aminonaphthalene, p,p′-diaminostilbenes, pyrenes, quaternary phenanthridine salts, 9-aminoacridines, p,p′-diaminobenzophenone imines, anthracenes, oxacarbocyanine, merocyanine, 3-aminoequilenin, perylene, bis-benzoxazole, bis-p-oxazolyl benzene, 1,2-benzophenazin, retinol, bis-3-aminopyridinium salts, hellebrigenin, tetracycline, sterophenol, benzimidazolylphenylamine, 2-oxo-3-chromen, indole, xanthen, 7-hydroxycoumarin, phenoxazine, calicylate, strophanthidin, porphyrins, triarylmethanes and flavin. Individual fluorescent compounds which have functionalities for linking to an element desirably detected in an apparatus or assay of the invention, or which can be modified to incorporate such functionalities include, e.g., dansyl chloride; fluoresceins such as 3,6-dihydroxy-9-phenylxanthydrol; rhodamineisothiocyanate; N-phenyl 1-amino-8-sulfonatonaphthalene; N-phenyl 2-amino-6-sulfonatonaphthalene; 4-acetamido-4-isothiocyanato-stilbene-2,2′-disulfonic acid; pyrene-3-sulfonic acid; 2-toluidinonaphthalene-6-sulfonate; N-phenyl-N-methyl-2-aminoaphthalene-6-sulfonate; ethidium bromide; stebrine; auromine-0,2-(9′-anthroyl)palmitate; dansyl phosphatidylethanolamine; N,N′-dioctadecyl oxacarbocyanine: N,N′-dihexyl oxacarbocyanine; merocyanine, 4-(3′-pyrenyl)stearate; d-3-aminodesoxy-equilenin; 12-(9′-anthroyl)stearate; 2-methylanthracene; 9-vinylanthracene; 2,2′(vinylene-p-phenylene)bisbenzoxazole; p-bis(2- -methyl-5-phenyl-oxazolyl))benzene; 6-dimethylamino-1,2-benzophenazin; retinol; bis(3′-aminopyridinium) 1,10-decandiyl diiodide; sulfonaphthylhydrazone of hellibrienin; chlorotetracycline; N-(7-dimethylamino-4-methyl-2-oxo-3-chromenyl)maleimide; N-(p-(2benzimidazolyl)-phenyl)maleimide; N-(4-fluoranthyl)maleimide; bis(homovanillic acid); resazarin; 4-chloro-7-nitro-2,1,3-benzooxadiazole; merocyanine 540; resorufin; rose bengal; and 2,4-diphenyl-3(2H)-furanone. Many fluorescent tags are commercially available from SIGMA chemical company (Saint Louis, Mo.), Molecular Probes, R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersberg, Md.), Fluka Chemica-Biochemika Analytika (Fluka Chemie AG, Buchs, Switzerland), and Applied Biosystems (Foster City, Calif.) as well as other commercial sources known to one of skill.

In an alternative embodiment, the probes may further comprise one or more crosslinking compounds. There are extensive methodologies for providing crosslinking upon hybridization between the probe and the target to form a covalent bond. Conditions for activation may include photonic, thermal, and chemical, although photonic is the primary method, but may be used in combination with the other methods of activation. Therefore, photonic activation will be primarily discussed as the method of choice, but for completeness, alternative methods will be briefly mentioned.

The probes will have from 1 to 5 crosslinking agents, more usually from about 1 to 3 crosslinking agents. The crosslinking agents must be capable of forming a covalent crosslink between the probe and target sequence, and will be selected so as not to interfere with the hybridization. In a preferred embodiment, the crosslinking agents in the probe will be positioned across from a thymine (T), cytosine (C), or uracil (U) base in the target sequence.

For the most part, the compounds that are employed for crosslinking will be photoactivatable compounds that can form covalent bonds with a base, particularly a pyrimidine. These compounds will include functional moieties, such as coumarin, as present in substituted coumarins, furocoumarin, isocoumarin, bis-coumarin, psoralen, etc.; quinones, pyrones, α,β-unsaturated acids; acid derivatives, e.g., esters; ketones; nitriles; azido compounds, etc. A large number of functionalities are photochemically active and can form a covalent bond with almost any organic moiety. These groups include carbenes, nitrenes, ketenes, free radicals, etc. One can provide for a scavenging molecule in the bulk solution, normally excess non-target nucleic acid, so that probes that are not bound to a target sequence will react with the scavenging molecules to avoid non-specific crosslinking between probes and target sequences. Carbenes can be obtained from diazo compounds, such as diazonium salts, sulfonylhydrazone salts, or diaziranes. Ketenes are available from diazoketones or quinone diazides. Nitrenes are available from aryl azides, acyl azides, and azido compounds. For further information concerning photolytic generation of an unshared pair of electrons, see Schoenberg, Preparative Organic Photochemistry, 1968.

Another class of photoactive reactants are inorganic/organometallic compounds based on any of the d- or f-block transition metals. Photoexcitation induces the loss of a ligand from the metal to provide a vacant site available for substitutions. Suitable ligands include nucleotides. For further information regarding the photosubstitution of these compounds, see Geoffrey and Wrighton, Organometallic Photochemistry, 1979.

In one preferred embodiment, the crosslinking agent comprises a coumarin derivative as described in co-pending U.S. patent application Ser. No. 09/390,124 and in U.S. Pat. No. 6,005,093, the disclosures of which are incorporated herein in their entirety. Briefly, with this embodiment the probes of the present invention benefit from having one or more photoactive coumarin derivatives attached to a stable, flexible, (poly)hydroxy hydrocarbon backbone unit. Suitable coumarin derivatives are derived from molecules having the basic coumarin ring system, such as the following: (1) coumarin and its simple derivatives; (2) psoralen and its derivatives, such as 8-methoxypsoralen or 5-methoxypsoralen (at least 40 other naturally occurring psoralens have been described in the literature and are useful in practicing the present invention); (3) cis-benzodipyrone and its derivatives; (4) trans-benzodipyrone and its derivatives; and (5) compounds containing fused coumarin-cinnoline ring systems. All of these molecules contain the necessary crosslinking group (an activated double bond) to crosslink with a nucleotide in the target strand.

Another preferred embodiment utilizes the aryl-olefin derivatives as the crosslinking agent, as described in U.S. patent application Ser. No. 09/189,294 and corresponding U.S. Pat. No. 6,303,799, the disclosures of which are incorporated herein in their entirety. In this embodiment, the double bond of the aryl-olefin unit is a photoactivatable group that covalently crosslinks to suitable reactants in the complementary strand. Thus, the aryl-olefin unit serves as a crosslinking moiety and is attached via a linker to a suitable backbone moiety incorporated into the probe sequence.

The probes may be prepared by any convenient method, most conveniently synthetic procedures, where the crosslinker-modified nucleotide is introduced at the appropriate position stepwise during the synthesis. Alternatively, the crosslinking molecules may be introduced onto the probe through photochemical or chemical monoaddition. The above patent disclosures provide specific teachings regarding the incorporation of coumarin and aryl-olefin derivatives, which are incorporated by reference herein. Linking of various molecules to nucleotides is well known in the literature and does not require description here. See, for example, Oligonucleotides and Analogues: A Practical Approach, Echstein (Ed.), 1991.

The probe and target will be brought together in an appropriate medium and under conditions that provide for the desired stringency to provide an assay medium. Therefore, usually buffered solutions will be employed, employing chemicals, such as citrate, sodium chloride, Tris, EDTA, EGTA, magnesium chloride, etc. See, for example, Sambrook et al., Molecular Cloning: A Laboratory Manual, 1988, for a list of various buffers and conditions, which is not an exhaustive list. Solvents may be water, formamide, DMF, DMSO, HMP, alkanols, and the like, individually or in combination, usually aqueous solvents. Temperatures may range from ambient to elevated temperatures, usually not exceeding about 100° C., more usually not exceeding about 90° C. Usually, the temperature for photochemical and chemical crosslinking will be in the range of about 20 to 70° C. For thermal crosslinking, the temperature will usually be in the range of about 70 to 120° C.

The amount of target nucleic acid in the assay medium will generally range from about 0.1 yoctomole to about 100 picomoles, more usually 1 yoctomole to 10 picomoles. The concentration of sample nucleic acid will vary widely depending on the nature of the sample. Concentrations of sample nucleic acid may vary from about 0.01 femtomolar to 1 micromolar. Similarly, the ratio of probe to target nucleic acid in the assay medium may vary, or be varied widely, depending upon the amount of target in the sample, the number and types of probes included in the probe mixture, the nature of the crosslinking agent, the detection methodology, the length of the complementarity region(s) between the probe(s) and the target, the differences in the nucleotides between the target and the probe(s), the proportion of the target nucleic acid to total nucleic acid, the desired amount of signal amplification, the incorporation of crosslinking agents, or the like. The probe(s) may be about at least equimolar to the target but are usually in substantial excess. Generally, the probe(s) will be in at least 10-fold excess, and may be in 10⁶-fold excess, usually not more than about 10¹²-fold excess, more usually not more than about 10⁹-fold excess in relation to the target. The ratio of capture probe(s) to reporter probe(s) in the probe mixture may also vary based on the same considerations.

Conveniently the stringency will employ a buffer composed of about 1× to 10×SSC or its equivalent. The solution may also contain a small amount of an innocuous protein, e.g., serum albumin, β-globulin, etc., generally added to a concentration in the range of about 0.5 to 2.5%. DNA hybridization may occur at elevated temperature, generally ranging from about 20 to 70° C., more usually from about 25 to 60° C. The incubation time may be varied widely, depending upon the nature of the sample, generally being at least about 5 minutes and not more than 6 hours, more usually at least about 10 minutes and not more than 2 hours.

In the crosslinking embodiment, after sufficient time for hybridization to occur, the crosslinking agent may be activated to provide crosslinking. As noted previously above, the activation may involve illumination, heat, chemical reagent, or the like, and will occur through actuation of an activator, e.g., a means for introducing a chemical agent into the medium, a means for modulating the temperature of the medium, a means for irradiating the medium, and the like. If the activatable group is a photoactivatablc group, the activator will be an irradiation means where the particular wavelength that is employed may vary from about 250 to 650 nm, more usually from about 300 to 450 nm. The illumination power will depend upon the particular reaction and may vary in the range of about 0.5 to 250 W. Activation may then be initiated immediately, or after a short incubation period, usually less than 1 hour, more usually less than 0.5 hour. With photoactivation, usually extended periods of time will be involved with the activation, where incubation is also concurrent. The photoactivation time will usually be at least about 1 minute and not more than about 2 hours, more usually at least about 5 minutes and not more than about 1 hour.

The purpose of introducing the covalent crosslink between the probes and target DNA is to raise effectively the Tm of the complex above that attained by hydrogen bonding alone. This property allows wash steps to be performed at greater stringency than under initial hybridization conditions, thereby markedly reducing non-specific binding. Thus, the methods of the present invention provide hybridization complexes in which the probe(s) and target sequence(s) are covalently linked to one another, not just hydrogen bonded together. Therefore, harsher conditions that will disrupt any undesirable, nonspecific background binding, but will not break the covalent bond(s) linking the probe to its target sequence, may be employed. For example, washes with urea solutions or alkaline solutions could be used. Heat could also be used. Accordingly, with this embodiment the covalent linkage provides for a significant improvement in the signal-to-noise ratio of the assay.

As described above, high-stringency conditions for the washing step generally employ low ionic strength and high temperature, or alternatively a denaturing agent, such as formamide. In a preferred embodiment, the wash conditions are 1×SSC/0.1% Tween 20 at room temperature (20-25° C.). In another preferred embodiment, the wash conditions are 50% formamide/0.5% Tween 20/0.1×SSC at room temperature (20-25° C.).

After crosslinking of the hybridized probes in the probe mixture, if such crosslinking agents are present, the label(s) incorporated into the probe(s) may be detected. As noted above, a number of different labels that can be used with the probes are known in the art. In the preferred embodiment, one or more capture probes having as a label a member of a specific binding pair, e.g., biotin, are combined with one or more reporter probes having a label that provides a detectable signal. In a preferred embodiment, the reporter probe is polyfluoresceinated to provide for increased signal generation. One may also use a substrate such as AttoPhos, as described herein, or other substrates that produce fluorescent products. With the present invention, the same sample can be contacted with different probe mixtures in different wells of the same microtiter plate in order to assay concurrently for polymorphisms such as SNPs as well as gene dosage abnormalities such as deletions and duplications.

In an alternative embodiment, the capture or reporter probes described herein may be linked covalently to a solid support prior to performance of the assay. In one such embodiment, a micro-formatted multiplex or matrix device may be used (e.g., DNA chips) (Barinaga, Science 1991; 253:1489; Bains, Bio/Technology 1992; 10:757-8). These methods usually attach specific DNA sequences to very small specific areas of a solid support, such as micro-wells of a DNA chip. In one variant, the assay is adapted to solid phase arrays for the rapid and specific detection of multiple polymorphisms of interest. A plurality of capture probes directed to a plurality of polymorphisms can be linked to a solid support and hybridized with a sample and corresponding sets of reporter probes. In this manner, the hybridization and subsequent detection of the corresponding reporter probes will be indicative of the presence or absence of the polymorphism at each site included in the array.

Exemplary solid supports include glass, plastics, polymers, metals, metalloids, ceramics, organics, etc. Using chip masking technologies and photoprotective chemistry it is possible to generate ordered arrays of nucleic acid probes. These arrays, which are known, e.g., as “DNA chips,” or as very large scale immobilized polymer arrays (“VLSIPS™” arrays) can include millions of defined probe regions on a substrate having an area of about 1 cm² to several cm², thereby incorporating sets of from a few to millions of probes.

The construction and use of solid phase nucleic acid arrays to detect target nucleic acids is well described in the literature. See, Fodor et al., Science 1991; 251:767-777; Sheldon et al., Clin. Chem. 1993; 39(4):718-9; Kozal et al., Nat. Med. 1996; 2(7): 753-9; and Hubbell U.S. Pat. No. 5,571,639. See also, Pinkel et al. PCT/US95/16155 (WO 96/17958). In brief, a combinatorial strategy allows for the synthesis of arrays containing a large number of probes using a minimal number of synthetic steps. For instance, it is possible to synthesize and attach all possible DNA 8 mer oligonucleotides (65,536 possible combinations) using only 32 chemical synthetic steps. In general, VLSIPS™ procedures provide a method of producing 4ⁿ different oligonucleotide probes on an array using only 4n synthetic steps.

Light-directed combinatorial synthesis of oligonucleotide arrays on a glass surface is performed with automated phosphoramidite chemistry and chip masking techniques similar to photoresist technologies in the computer chip industry. Typically, a glass surface is derivatized with a saline reagent containing a functional group, e.g., a hydroxyl or amine group blocked by a photolabile protecting group. Photolysis through a photolithogaphic mask is used selectively to expose functional groups which are then ready to react with incoming 5′-photoprotected nucleoside phosphoramidites. The phosphoramidites react only with those sites which are illuminated (and thus exposed by removal of the photolabile blocking group). Thus, the phosphoramidites only add to those areas selectively exposed from the preceding step. These steps are repeated until the desired array of sequences have been synthesized on the solid surface.

A 96-well automated multiplex oligonucleotide synthesizer (A.M.O.S.) has also been developed and is capable of making thousands of oligonucleotides (Lashkari et al., PNAS 1995; 93:7912). Existing light-directed synthesis technology can generate high-density arrays containing over 65,000 oligonucleotides (Lipshutz et al., BioTech. 1995; 19:442.

Combinatorial synthesis of probe sequences at different locations on the array is determined by the pattern of illumination during synthesis and the order of addition of coupling reagents. Monitoring of hybridization of reporter probes to the array is typically performed with fluorescence microscopes or laser scanning microscopes. In addition to being able to design, build and use probe arrays using available techniques, one of skill is also able to order custom-made arrays and array-reading devices from manufacturers specializing in array manufacture. For example, Affymetrix Corp., in Santa Clara, Calif. manufactures DNA VLSIP™ arrays.

The following examples are offered by way of illustration and not by way of limitation. All references cited herein are specifically incorporated by reference.

### EXAMPLES

### Example 1

Gene Dosage and SNP Assay from Gene Conversion Mutations: Parallel Assessment of Four Common SNPs. Gene Deletions and Duplications in CYP2D6 Gene

Pharmacogenetics is an area of emerging clinical importance based on the recognition that genetic polymorphism affecting function of proteins involved in drug metabolism and receptor binding kinetics have profound effects on individual medication response. The most significant pharmacogenetic loci to date are those of the cytochrome P450 group, whose protein products are responsible for the activation or degradation of the majority of drugs (Linder M W, Valdes R Jr. Pharmacogenetics in the practice of laboratory medicine. Mol Diagn. 1999;4:365-79., Meyer U A, Zanger U M. Molecular mechanisms of genetic polymorphisms of drug metabolism. Annu Rev Pharmacol Toxicol. 1997;37:269-96). The cytochrome P450 loci have emerged through gene copying and subsequent divergent natural selection. These genes share strong homology with each other and usually with non-functioning pseudogenes as well. Cross-hybridization with homologous sequences confounds standard hybridization and PCR-based methodologies. To date, high-throughput, cost-effective methods for assaying these loci have not been produced.

The CYP2D6 gene represents the most clinically important pharmacogenetic locus as yet defined (Sachse C, Brockmoller J, Bauer S, Roots I. Cytochrome P450 2D6 variants in a Caucasian population: allele frequencies and phenotypic consequences. Am J Hum Genet. 1997;60:284-95. Marez D, Legrand M, Sabbagh N, Guidice J M, Spire C, Lafitte J J, Meyer U A, Broly F. Polymorphism of the cytochrome P450 CYP2D6 gene in a European population: characterization of 48 mutations and 53 alleles, their frequencies and evolution. Pharmacogenetics 1997;7:193-202. Scarlett L A, Madani S, Shen D D, Ho R J. Development and characterization of a rapid and comprehensive genotyping assay to detect the most common variants in cytochrome P450 2D6. Pharm Res. 2000;17:242-6. Gaedigk A, Gotschall R R, Forbes N S, Simon S D, Kearns G L, Leeder J S. Optimization of cytochrome P4502D6 (CYP2D6) phenotype assignment using a genotyping algorithm based on allele frequency data. Pharmacogenetics. 1999;9:669-82). This gene product is responsible for the metabolism of about 25% of the commonly prescribed drugs today, including most of the beta blockers and antiarrhythmic drugs in use and about half of the tricyclic and selective serotonin reuptake inhibitor antidepressants. Both low and enhanced functioning alleles have been described attributable to inactivating SNPs or gene deletions, or gene duplications from 2 to 10 copies, respectively. Inheritance of two inactivating mutations is associated with the “poor metabolizer” phenotype, comprising toxicity due to accumulation of active compounds and lack of drug response attributable to failure of activation of prodrug. The “ultra-metabolizer” phenotype results from duplication alleles inherited in a dominant fashion producing increased gene dosage and consequent under-dosing of many important drugs. The incidence of both poor and ultra-metabolizers is estimated at about 5% of the American population each. To date, 53 alleles of CYP2D6 have been described, the majority functionally neutral. A total of four SNPs (designated *3, *4, *6, and *7) and a whole-gene deletion allele (designated *5) contribute about 98% of the poor metabolizer genotypes, while duplication alleles make up the entirety of the ultra-metabolizer alleles. Currently there is a great demand from the pharmaceutical industry for genotyping of subjects enrolled in clinical trials and it is anticipated that there will be future interest in genotyping subjects prior to initiation of certain medications.

The CYP2D6 locus is complex, having undergone serial duplication events resulting in the presence of two highly homologous sequences, CYP2D7 and CYP2D8 just upstream. Absent of selective pressures, CYP2D7 and CYP2D8 have accumulated mutations rendering them untranslated. These loci share greater than 90% identity with CYP2D6 complicating molecular diagnostics. Current genotyping assays are extremely problematic, relying on the generation of long PCR products for SNP analysis and Southern blotting for dosage analysis. Chip-based oligonucleotide hybridization assays suffer from inaccuracy presumably due to crosshybridization with the pseudogenes. Photocrosslinking oligonucleotide hybridization technology has been shown to reliably discriminate the factor V Leiden and hereditary hemochromatosis HFE C282Y and H63D single nucleotide polymorphisms in a high-throughput format (Zehnder J, Van Atta R, Jones C, Sussmann H, Wood M. Cross-linking hybridization assay for direct detection of factor V Leiden mutation. Clin Chem 1997;43:1703-8; Wylenzek C, Engelmann M, Holten D, Van Atta R, Wood M, Gathof B. Evaluation of a nucleic acid-based cross-linking assay to screen for hereditary hemochromatosis in healthy blood donors. Clin Chem 2000;46:1853-5.). It has been subsequently adapted to effectively determine gene dosage at the Prader-Willi/Angelman syndrome locus at 15q11-q13 (Peoples R, Weltman H, Van Atta R, Wang J, Wood M, Ferrante-Raimondi M, Cheng P, et al. High-Throughput Detection of Submicroscopic Deletions and Methylation Status at 15 q11-q13 by a Photo-Cross-Linking Oligonucleotide Hybridization Assay. Clinical Chemistry 2002;48:in press). By allowing high-stringency washing of covalently bound photocrosslinked probe target complexes, non-specific hybridization is minimized and linearity between template quantity and signal is maintained, affording accurate assessment of relative target amounts. The technology is ideally suited for concurrent assessment of SNP mutations and gene dosage due to the standardization of wash stringency afforded by the probe-target crosslinking. This methodology has been applied to development of an assay interrogating the four common SNP alleles in parallel with assessment of overall locus copy number. A new method is described allowing for target specification through the reporter probe function obviating the need for PCR-based target selection and mitigating the effects of potentially cross-hybridizing loci.

Oligonucleotide hybridization-based detection of the common CYP2D6 SNPs is typically confounded by allele specific capture probes demonstrating cross-reactivity with the “pseudogene” loci. Therefore, the present assay was designed to take advantage of the potential of the reporter probes of the present invention to “build in” locus-specificity while the capture probe confers specificity for the particular allele. A sequence of almost 2 kb was identified over a region of the CYP2D6 gene containing all four SNP sites as well as a complement of 20 potential CYP2D6-specific, crosslinker-containing reporter sequences. Each of these sequences included a minimum of 20% site-discriminating or “locus-specific” nucleotides, i.e. nucleotides distinguishing the CYP2D6 gene from each of CYP2D7 and CYP2D8. Bifluoresceinated reporter probes were synthesized and used in conjunction with two capture probes sharing identity between CYP2D6, CYP2D7 and CYP2D8. Using long PCR products specific for each of CYP2D6, CYP2D7 and CYP2D8 as template, photocrosslinking assays were performed as described (Zehnder J, Van Atta R, Jones C, Sussmann H, Wood M. Cross-linking hybridization assay for direct detection of factor V Leiden mutation. Clin Chem 1997;43:1703-8; Wylenzek C, Engelmann M, Holten D, Van Atta R, Wood M, Gathof B. Evaluation of a nucleic acid-based cross-linking assay to screen for hereditary hemochromatosis in healthy blood donors. Clin Chem 2000;46:1853-5.) using the common capture and potentially CYP2D6-specific reporter probes. As the DNA is size-fragmented by enzymatic digestion, the pre-assay boiling time is reduced to 5 minutes for the sole purpose of target denaturation. Results led to the selection of a panel of 11 reporter probes yielding excellent signal-to-background ratios and conferring CYP2D6 specificity. The ratio of absolute signal obtained using the described probe sets with the CYP2D6 template relative to each of the CYP2D7 and CYP2D8 PCR product templates was derived. Reporter probes whose ratios were greater than 90% for both CYP2D7 and CYP2D8 signals as the denominator were included in this panel.

Four SNP-specific capture probe pairs and an invariant CYP2D6 dosage capture probe can then each be used in conjunction with the set of CYP2D6-reporter probes in photocrosslinking assays to generate a comprehensive genotype of the CYP2D6 locus. Reporter probes will be modified by addition of the polyfluorescein moiety for greater signal generation as described for the 15q11-q 13 assay (Peoples R, Weltman H, Van Atta R, Wang J, Wood M, Ferrante-Raimondi M, Cheng P, et al. High-Throughput Detection of Submicroscopic Deletions and Methylation Status at 15q11-q13 by a Photo-Cross-Linking Oligonucleotide Hybridization Assay. Clinical Chemistry 2002;48:in press). The lone deviation from that protocol comprises the substitution of Eag I, Bam HI, and HinC II for Hpa II in the enzyme digestion step to generate fragments of 2098 bps from the CYP2D6 locus and 1843 bps from the ANK2 locus.

Experimentally selected reporter probes and designed capture probes are included below: The SNP-specific probes are designated by the * system. This assay makes use of a modification of the photocrosslinking capture probe system designed to incresase the flexibility of capture probe design. Photocrosslinking optimally proceeds when the XLnt moiety is opposing a T residue. Therefore, crosslinking may be accomplished through a secondary mechanism employing the use of a flanking probe or probes designed to be complementary to sequence immediately contiguous to the capture probe. The flanking probes can crosslink to target, while crosslinking of flnaking probes to capture probes is mediated through the use of tailed structures as illustrated. X″ denotes the crosslinks and “ ” the SNP site. The center probe is labeled with biotin for probe capture.

[image]

This design allows allele-specific probe design to proceed independent of the need for viable crosslinking sites in the immediate region of the mutation, a challenge in particularly GC-rich areas.

Sequences given below are obtained from GenBank sequences M33388 (CYP2D6) and ACC004057 (ANK2). “Δ7” an “Δ8” refer to the number of nucleotide differences between the corresponding CYP2D6 and CYP2D7, or CYP2D6 and CYP2D8 genes, respectively. “X” denotes the XLnt crosslinking nucleotide.

CYP2D6 gene

| | GB number | Probe sequence | Δ7 | Δ8 |
|---|---|---|---|---|
| REP | 2695-2714 | AXACAGATTTCCGTGGACC | 4 | 4 |
| REP | 2732-275 | TAGTCCGAGCTGGGCAGAXA | 5 | 3 |
| REP | 2753-2770 | GGCGCGGGGTCGTGGAXA | 3 | 3 |
| REP | 2804-2824 | AXAAACCACCTGCACTAGGGA | 3 | 4 |
| REP | 2929-2948 | AXTCCGGTGTCGAAGTGGGG | 6 | 6 |
| REP | 3073-3092 | GAGCAAGGTGGATGCACAXA | 4 | 6 |
| REP | 3136-3153 | AXACCAGGGGGAGCATAG | 6 | 4 |
| REP | 3166-3185 | TGGTGGATGGTGGGGCTAXT | 4 | 6 |
| REP | 3686-3704 | GGACTGGGGCCTCGGAAXA | 2 | 8 |
| REP | 3850-3870 | GTACCTCCTATCCACGTCAXA | 5 | 5 |
| REP | 3102-3120 | CTGTGACCAGCTGGACAXA | 3 | 2 |
| *3 CAP | 4168 | CTGAGCAC(A/-)GGATGACC | | |
| Flank | | AXAGGCTTTCCTGACCCAGCTGG | | |
| | | ATGAGCTGCTAA-tail | | |
| Flank | | tail-TGGGACCCAGCCCAGCCCCC | | |
| | | CCCAXA | | |
| *4 CAP | 3465 | CACCCCCA(G/A)GACGCCCC | | |
| Flank | | GXAGGCGACCCCTTACCCGCATCT | | |
| | | CC-tail | | |
| Flank | | tail-TTTCGCCCCAACGGTCTC | | |
| | | TTGGACAXA | | |
| *6 CAP | 3326 | TGGAGCAG(T/-)GGGTGACC | | |
| Flank | | CXACTTGGGCCTGGGCAAGAAGT | | |
| | | CGC-tail | | |
| Flank | | tail-GAGGAGGCCGCCTGCCT | | |
| | | TTGTGCCGCCTTCGCCAXC | | |
| *7 CAP | 4554 | GATCCTAC(A/-)TCCGGATG | | |
| Flank | | AXAACCTGCGCCATAGTGGTGGC | | |
| | | TGACCTGTTCTCTGCCGGGATGGT | | |
| | | GACCACCTCGACCACGCTGGCCT | | |
| | | GGGGCCTCCTGCTCAT-tail | | |
| Flank | | tail- | | |
| | | TGCAGCGTGAGCCCATCTGGGAXA | | |
| Eag I | 2576 | | | |
| Bam HI | 4674 | | | |

ANK2 gene control

| | GB number | Probe sequence |
|---|---|---|
| CAP | 22213-22232 | AGTCATGTGAACTAGCTAXA |
| REP | 22283-22302 | AXAGGGTCCTGACCTCATGC |
| REP | 22329-22348 | AXATGGGGAGCCACCATAGA |
| REP | 22535-22554 | AXAATATCAGCAACATTCAC |
| REP | 22638-22657 | AXATACATTGCATCATCTAT |
| REP | 22700-22719 | AXACTCATAGCCTCTTCCCA |
| REP | 22866-22885 | AXATAGCACAGCCAATAAGC |
| REP | 22946-22965 | AXATAGCTGATCAACCAACT |
| REP | 23033-23052 | AXATGGACAGTTACAGGAAA |
| REP | 23064-23-83 | AXACTTTCTCCAGCACCCAA |
| REP | 23268-23287 | AXATGGGGGAAAGTGGCTTA |
| HinC II | 21757, 23600 | |

### EXAMPLE 2

A Photocrosslinking Oligonucleotide Hybridization Assay Assessing the Common Small and Large Deletions and Conversion Mutations of the SMN Genes at the Spinal Muscular Atrophy Locus at 5q12.2-g13.3

Autosomal recessive SMA occurs in 1/10,000 births and results in progressive motor weakness of variable severity associated with clinical sub-phenotypes I, II and III. The locus at 5q12.2-q13.3 comprises tandem inverted duplications of two roughly 500 kb DNA sequences of remarkably high homology (Scheffer H, Cobben J M, Matthijs G, Wirth B. Best practice guidelines for molecular analysis in spinal muscular atrophy. Eur J Hum Genet 2001;9:484-91. Feldkotter M, Schwarzer V, Wirth R, Wienker T F, Wirth B. Quantitative analyses of SMN 1 and SMN2 based on real-time lightCycler PCR: fast and highly reliable carrier testing and prediction of severity of spinal muscular atrophy. Am J Hum Genet. 2002;70:358-68). Absence of sequence specific to the telomeric, or functional, copy of the SMN gene (SMN1 or SMNtel) is causative in >95% of the defined cases of SMA. This absence of sequence is variably attributable to deletion of the SMNtel gene or conversion mutations conferring the SMNcen sequence at the SMNtel locus. The area has been intensely studied and 5 invariant, SMNtel-specific nucleotides in the 3′ end of the gene from intron 6 to exon 8 have been identified (Lefebvre S, Burglen L, Reboullet S, Clernont 0, Burlet P, Viollet L, Benichou B, et al. Identification and characterization of a spinal muscular atrophy-determining gene. Cell 1995;80:155-65. Burglen L, Lefebvre S, Clermont 0, Burlet P, Viollet L, Cruaud C, Munnich A, Melki J. Structure and organization of the human survival motor neurone (SMN) gene. Genomics 1996;32:479-82). Particularly, a single nucleotide substitution of T (centromeric) for C (telomeric) at exon 7 (+6 position) has been shown to alter RNA splicing excluding exon 7, producing a poorly functional protein (Lorson C L, Hahnen E, Androphy E J, Wirth B. A single nucleotide in the SMN gene regulates splicing and is responsible for spinal muscular atrophy. Proc Natl Acad Sci USA 1999;96:6307-11.). Analysis of sequence from subjects harboring “conversion alleles” in which SMN copy number is normal but exon 7 is skipped reveal that in these cases, all 4 site-defining nucleotides from intron 6 to intron 7 have adopted an SMNcen pattern, while the exon 8 (+245 position) nucleotide retains the SMNtel-specific G (Hahnen E, Schonling J, Rudnik-Schonebom S, Zerres K, Wirth B. Hybrid survival motor neuron genes in patients with autosomal recessive spinal muscular atrophy: new insights into molecular mechanisms responsible for the disease. Am J Hum Genet 1996;59:1057-65). Molecular diagnostic assays have generally involved PCR-based amplification and sequence analysis of these site-specifying nucleotides. While these assays do not differentiate the conversion from deletion mutations, they can confirm absence of functional SMNtel sequence. More problematic has been detection of carrier status, estimated at 1 in 50, in the U.S. population. Detection of mutant alleles in the presence of a normal homologue confounds non-quantitative detection. Several assays have been reported using quantitative PCR methodology for assay purposes, but to date, no non-amplified method can successfully identify carriers. Furthermore, larger deletions affecting both the telomeric and centromeric loci are associated with a more severe phenotype, making assessment of copy number for both telomeric and centromeric genes desirable.

The XLnt photocrosslinking oligonucleotide hybridization technology has been shown to reliably discriminate the factor V Leiden and hereditary hemochromatosis HFE C282Y and H63D single nucleotide polymorphisms (SNPs) in a high-throughput format (Zehnder J, Van Atta R, Jones C, Sussmann H, Wood M. Cross-linking hybridization assay for direct detection of factor V Leiden mutation. Clin Chem 1997;43:1703-8; Wylenzek C, Engelmann M, Holten D, Van Atta R, Wood M, Gathof B. Evaluation of a nucleic acid-based cross-linking assay to screen for hereditary hemochromatosis in healthy blood donors. Clin Chem 2000;46:1853-5). It has been subsequently adapted to effectively determine gene dosage at the Prader-Willi/Angelman syndrome locus at 15q11-q13 (Peoples R, Weltman H, Van Atta R, Wang J, Wood M, Ferrante-Raimondi M, Cheng P, et al. High-Throughput Detection of Submicroscopic Deletions and Methylation Status at 15q11-q13 by a Photo-Cross-Linking Oligonucleotide Hybridization Assay. Clinical Chemistry 2002;48:1844-50). By allowing high-stringency washing of covalently bound photocrosslinked probe target complexes, non-specific hybridization is minimized and linearity between template quantity and signal is maintained, affording accurate assessment of relative target amounts. An XLnt assay using direct hybridization-based detection in a high-throughput format for assessment of telomeric-specific SMN gene dosage would represent a profound improvement over existing techniques. The XLnt system has been adapted to allow complete SMNtel genotype-determination in the setting of highly homologous, potentially cross-hybridizing, sequences at the SMA locus at 5q12.2-q13.3. First, SMNtel-specific dosage determines functional SMN copy number, allowing rapid carrier screening. Secondly, a method has been developed utilizing separate capture and reporter probes affording interrogation of the single exon 8 G (telomeric pattern) allele downstream of SMNcen-specific sequence for assessment of presumptive conversion mutations yielding a hybrid gene. In parallel, dosage assessment of the entirety of SMNtel and SMNcen sequence is performed to yield a complete profile of the SMA locus. Such an assay can be performed in an automated, high-throughput fashion, offering the potential for rapid, comprehensive diagnosis of affected individuals.

An XLnt photocrosslinking assay assessing 1) dosage of the SMNtel gene carrying the functional exon 7 C allele, 2) overall SMN gene dosage, and 3) presence of the intron 6 through intron 7 “centromeric pattern” directly upstream of the exon 8 G allele is described. Four probe sets are used comprising a functional SMNtel-specific set, a common SMNtel/cen set, a SMNtel-SMNcen hybrid gene/conversion allele set and a dosage control set. The first set (SMNtel probe set) utilizes an allele-specific capture probe recognizing the functional exon 7 C allele (SMNtel-7 capture probe) and a set of 4 reporter probes complementary to sequence common to the centromeric and telomeric genes (SMNtel/cen reporter probes). The second set (SMN common probe set) uses the same 4 SMNtel/cen reporter probes described above and a capture probe drawn from common SMNtel/cen sequence-(SMNtel/cen capture probe). The third set (SMN hybrid probe set) comprises an allele-specific capture probe recognizing the “telomeric pattern” exon 8 G allele (SMNtel-8 capture probe) and a set of 4 SMNcen-specific reporter probes (SMNcen reporter probes) designed around the intron 6, exon 7 and intron 7 site-specifying nucleotides. A fourth probe set (ANK2 probe set) recognizes sequence from the ANK2 locus at 4q25 as an obligate two-copy dosage control. Subcloned PCR products of 849 bps containing the 5 invariant nucleotides defining each of the centromeric and telomeric SMN genes are used as templates in experiments assessing the optimum length for each of the SMNtel-specific capture and SMNcen-specific reporter probes, in terms of signal-to-noise ratio and allele (capture probe) or locus (reporter probes) specificity. Each of the biotinylated capture probes are 17 bps, and contains one of the coumarin-based photocrosslinking moieties in place of a nucleotide at the 5′ or 3′ end. Each of the reporter probes are 16 bps each, labeled with the polyfluorescein group as described and each contains a single photocrosslinking group at one of the 3′ or 5′ termini. Probe sequences and PCR primer sequences for the SMN and ANK2 genes are given below. Nucleotide numbers for SMN sequences conform to those of chromosome 5 clone CTC-340H12, GenBank accession number AC016554; those for the ANK2 intragenic sequence were obtained from clone B240N9, GenBank accession number ACC004057. An “X” denotes the substitution of the photocrosslinking nucleotide. Allele or site-specifying nucleotides are in boldface. Nucleotide numbers are given for the Pst I and Hph I restriction sites that will be used for generation of target fragments (see below).

| | | | |
|---|---|---|---|
| Probe type | nucleotide | Nucleotide number | Sequence |

| | | | |
|---|---|---|---|
| SMNtel-7 capture probe | Ex7(+6) C/T | 110698-110714 | ACAGGGTTTCAGACAXA |
| SMNtel/cen capture probe | | 110979-110995 | AXACATACTTTCACAAA |
| SMNtel-8 capture probe | Ex8(+245) G/A | 111427-111443 | AXAGACTGGGGTGGGGG |
| SMNtel/cen reporter probe | | 110890-110907 | AXAGAATTTTGATGCC |
| SMNtel/cen reporter probe | | 111142-111157 | AXAGGACATGGTTTAA |
| SMNtel/cen reporter probe | | 111302-111317 | AXATATCAAGTGTTGG |
| SMNtel/cen reporter probe | | 111359-111374 | AXAGTTATGTAATAAC |
| SMNcen reporter probe | In6(−45) G/A | 110651-110666 | TATCTATATCTATAXA |
| SMNcen reporter probe | Ex7(+6) C/T | 110699-110714 | CAGGGTTTTAGACAXA |
| SMNcen reporter probe | In7(+100) A/G | 110847-110862 | AXATGTTAGAAAGTTG |
| SMNcen reporter probe | In7(+214) A/G | 110963-110978 | GTTGGTTGTGTGGAXG |
| Forward SMN primer | | 110621-110640 | AACATCCATATAAAGCTATC |
| Reverse SMN primer | | 111470-111451 | CTGCGTCACCACCGTGCTGG |
| SMN Pst I site | | 110495 | |
| SMN Hph I site | | 111461 | |
| ANK2 capture probe | | 2325 1-23267 | AGAAAGGCATGGAGAXA |
| ANK2 reporter probe | | 22616-22631 | AXAGGGATAGAGTTGA |
| ANK2 reporter probe | | 22811-22826 | AXATTACATTTTCTAT |
| ANK2 reporter probe | | 22946-22961 | AXATAGCTGATCAACC |
| ANK2 reporter probe | | 23082-23097 | AXAGAGGGTATACTTT |
| Forward ANK2 primer | | 22563-22582 | CCTGGGCTGCAAGGTGTAAG |
| Reverse ANK2 primer | | 23520-23501 | CTGCAGGATGTCCAGGAAGA |
| ANK2 Pst I site | | 23515 | |
| ANK2 Hph I site | | 22531 | |

Performance of the microtiter-plate based photocrosslinking oligonucleotide hybridization assays has been described (Peoples R, Weltman H, Van Atta R, Wang J, Wood M, Ferrante-Raimondi M, Cheng P, et al. High-Throughput Detection of Submicroscopic Deletions and Methylation Status at 15q11-q13 by a Photo-Cross-Linking Oligonucleotide Hybridization Assay. Clinical Chemistry 2002;48:1844-50). Briefly, target DNA and probes are combined under denaturing conditions, solutions are neutralized and hybridization proceeds. The plate is exposed to UV light to allow crosslinking and the wells are washed at high-stringency using a magnetic capture system. Signal generation proceeds through sequential incubation with an anti-fluorescein alkaline-phospatase conjugate and the alkaline phosphatase substrate, AttoPhos. The fluorescent signal is then read in a fluorimeter. The lone deviation from the protocol set forth for the 15q11-q13 assay comprises the substitution of Pst I and Hph I for Hpa II in the enzyme digestion step to generate fragments of 966 bps from the SMNtel and SMNcen loci and 984 bps from the ANK2 locus. As the DNA is size-fragmented by enzymatic digestion, the pre-assay boiling time is reduced to 5 minutes for the sole purpose of target denaturation. Processed samples are aliquoted into each of 6 wells and assayed with each of the three probe sets in duplicate. Control samples comprise SMNtel, SMNcen and ANK2 PCR products with concentrations adjusted to reflect normal 2-copy SMNtel and SMNcen dosage for use as a positive control and a negative control containing all components of the sample processing solution absent DNA.

Interpretation of data proceeds as follows: The mean signal is obtained for each sample with each probe set and corrected for background by subtraction of a negative control result. Sample values are then normalized to the result from the positive control for that probe set. Ratios are determined for the SMNtel-to-ANK2 values (ratio 1), SMN common-to-ANK2 (ratio II) values and the SMN hybrid-to-ANK2 values (ratio III). The first ratio will reflect dosage of the functional SMNtel genes, while the second determines the overall SMN gene copy number. The third ratio reflects presence of the hybrid SMNtel-SMNcen gene produced by conversion mutations. Taken together, the three values provide a profile of the SMA region. The following table illustrates some hypothetical profiles and the corresponding genotypes and phenotypes.

| | | | | Con- | | |
|---|---|---|---|---|---|---|
| | | | | ver- | Small | Conversion |
| | | | Large | sion | deletion/ | mutation/ |
| Geno- | Wild- | Small | dele- | muta- | large | small |
| type | type | deletion | tion, | tion | deletions | deletion |
| Ratio I | 1.0 | 0.5 | 0.5 | 0.5 | 0. | 0 |
| Ratio II | 2.0 | 1.5 | 1.0 | 2.0 | 0.5 | 1.5 |
| Ratio III | 0 | 0 | 0 | 1.0 | 0 | 1.0 |
| Pheno- | Un- | Carrier | Carrier | Carri- | SMA I | SMA II |
| type | affected | | | er | | or III |

AS the deletion and conversion mutations represent over 90% of the alleles in most populations, and close to 99% of affected individuals harbor at least one of these alleles, an assay using only the SMNtel and ANK2 probe sets would be of potential utility in a screening program. This particular assay is an example of using allele-specific dosage determination for carrier screening.

### Example 3

Application of Reporter Probe Specificity Methodology for Detection of Chromosomal Rearrangements Including Balanced and Unbalanced Translocations, and Inversion.

An extension of this methodology is the special case of chromosomal translocation detection with or without quantification. In this example, the “homologous sequences” comprise a given sequence in close proximity to a variably present chromosomal breakpoint such that contiguous sequence is either that of the wild-type chromosome, or of unique genetic material translocated from another chromosome arm.

[image]

The translocation chromosome, then, is chimeric, in that sequence from one chromosome has been substituted in a specific place with sequence from another. The detection method will then involve using capture probes recognizing identical sequences from each of the wild-type and translocation chromosomes, with “locus-specifying” reporter probes that recognize only one of the two chromosomes. Sample preparation methods must include the generation of target including the potential breakpoint region and flanking regions complementary to these probes.

[image]

The translocation chromosome may be present in the germline or the result of a somatic mutation, detectable only in certain tissues and at less than normal haploid dosage. The translocation may be “balanced”, in the setting of reciprocal chromosomal arm exchange events in which two translocation chromosomes are formed with the normal complement of genes present in standard amounts. The translocation may be “unbalanced”, in which some chromosomal material is either lost or duplicated.

There are multiple clinical applications of this method, including detection and quantitation of the Philadelphia chromosome translocation in CML that results in a fusion gene created by joining 5′ sequences of the BCR gene on chromosome 22 with 3′ sequences of the ABL gene from chromosome 9. Detection of this gene is critical for determining chemosensitivity to the tyrosine kinase inhibitor, Imatinib mesylate (STI571 or Gleevec/Glivec, Novartis), while accurate quantitation of the gene product is necessary for monitoring therapeutic response and identifying relapse (Kantarjian H M, Cortes J E, O'Brien S, Giles F, Garcia-Manero G, Faderl S, Thomas D, Jeha S, Rios M B, Letvak L, Bochinski K, Arlinghaus R, Talpaz M. Imatinib mesylate therapy in newly diagnosed patients with Philadelphia chromosome-positive chronic myelogenous leukemia: high incidence of early complete and major cytogenetic responses. Blood 2003;10:97-100; Wang L, Pearson K, Pillitteri L, Ferguson J E, Clark R E. Serial monitoring of BCR-ABL by peripheral blood real-time polymerase chain reaction predicts the marrow cytogenetic response to imatinib mesylate in chronic myeloid leukaemia. Br J Haematol 2002;118:771-7).

Another application is the detection of gene rearrangements, such as the inversion mutation responsible for most of the cases of Hemophilia A due to factor VIII deficiency. As the rearrangements are reciprocal, detection of them is extremely problematic (Bowen D J, Keeney S. Unleashing the long-distance PCR for detection of the intron 22 inversion of the factor VIII gene in severe haemophilia A. Thromb Haemost 2003;89:201-2).

An assay for detection and quantitation of the BCR-ABL oncogene transcript in Philadelphia chromosome+CML

The three most common BCR-ABL fusion genes result from translocations bringing into contiguity the BCR gene up to exons1, 13 or 14, and 19 at the 5′ end, and the ABL gene from exon 2 at the 3′ end; these transcripts result in protein products of 185, 210 and 230 kD, respectively (Martinelli G, Terragna C, Amabile M, Montefusco V, Testoni N, Ottaviani E, et al. Alu and translisin recognition site sequences flank translocation sites in a novel type of chimeric BCR-ABL transcript and suggest a possible general mechanism for BCR-ABL breakpoints. Haematologica 2000; 85:40-6; Testoni N, Martinelli G, Farabegoli P, Zaccaria A, Amabile M, Raspadori D, et al. A new method of “in cell RT-PCR” for the detection of bcr-abl transcript in chronic myeloid leukemia patients. Blood 1996; 87:3822-7). The proposed assay uses RNA isolated from peripheral blood or bone marrow aspirates as a template for quantitative detection of the four common BCR-ABL translocation products and the intact ABL gene. The assay will use the XLnt solution-based assay described for the CYP2D6 and SMN assays above (Zehnder J, Van Atta R, Jones C, Sussmann H, Wood M. Cross-linking hybridization assay for direct detection of factor V Leiden mutation. Clin Chem 1997;43:1703-8; Wylenzek C, Engelmann M, Holten D, Van Atta R, Wood M, Gathof B. Evaluation of a nucleic acid-based cross-linking assay to screen for hereditary hemochromatosis in healthy blood donors. Clin Chem 2000;46:1853-5; Peoples R, Weltman H, Van Atta R, Wang J, Wood M, Ferrante-Raimondi M, Cheng P, et al. High-Throughput Detection of Submicroscopic Deletions and Methylation Status at 15q11-q 13 by a Photo-Cross-Linking Oligonucleotide Hybridization Assay. Clinical Chemistry 2002;48:1844-50). This system provides for extreme quantitivity and sensitivity based on the ability of the covalently attached probe:target complexes to withstand higher stringency was conditions than in the absence of crosslinking. Total RNA will be extracted from clinical samples; as RNA templates are shorter and less complex than genomic DNA, size-fractionation of the template with restriction enzymes and template denaturation as described for the former assays is not necessary. Extracted RNA will be aliquotted into each of 5 separate wells of a 96-well microtitre plate. A hybridization solution will be added containing a common biotinylated capture probe designed from sequences complementary to the ABL gene exon 2. For each of the five wells, a discrete reporter probe set will be added recognizing sequences from each of the following sites: BCR exon 1; BCR exon 13; BCR exon 14; BCR exon 19; and ABL exon 1. Using a minimum of three reporter probes polyfluoresceinated for signal elaboration as described (Peoples R, Weltman H, Van Atta R, Wang J, Wood M, Ferrante-Raimondi M, Cheng P, et al. High-Throughput Detection of Submicroscopic Deletions and Methylation Status at 15q11-q13 by a Photo-Cross-Linking Oligonucleotide Hybridization Assay. Clinical Chemistry 2002;48:1844-50) the assay is predicted to yield results using a minimum of 1-5 ug per well of total RNA without the need for target amplification. This is a clinically realistic amount to be obtained from less than 0.5 mls of peripheral blood. The photocrosslinking chemistry is effective for both RNA and DNA templates, removing the necessity of transcribing RNA into DNA. Assay performance has been described (Peoples R, Weltman H, Van Atta R, Wang J, Wood M, Ferrante-Raimondi M, Cheng P, et al. High-Throughput Detection of Submicroscopic Deletions and Methylation Status at 15q11-q13 by a Photo-Cross-Linking Oligonucleotide Hybridization Assay. Clinical Chemistry 2002;48:1844-50).

Probes are designed to be from 15 to 20 base pairs and to incorporate, one (reporter probes) or two (capture probe) crosslinking sites, situated at the 3′ or 5′ terminus, or both. Furthermore, crosslinking occurs most effectively opposing thymidine residues. Probe sequences are from GenBank, accession numbers U07563 (ABL gene) and U07000 (BCR gene).

| | | |
|---|---|---|
| ABL exon 2 cap- | 50024-50004 | AXATCATACAGTGCAACGAXA |
| ture | | |
| ABL EX 1A | 37840-37826 | GGCAGATCTCCAAXA |
| | 37876-37861 | AXAGCCCCTTCTTGGA |
| | 37903-37891 | CTTCCAGATAAXA |
| BCR EX 1 | 16126-16107 | AXCATGCGGTAGGTGGTGGG |
| | 16263-16234 | TGAGATGGTGGCCTCGGAXA |
| | 16304-16289 | GXAGGGGCCCTCGCCA |
| BCR EX 13 | 123614-123596 | CAGGGAGAAGCTTCTGAXA |
| | 123667-123640 | AXAGTCTACACGAGTTGG |
| | 123694-123675 | TTATTGATGGTCAGCGGTXA |
| BCR EX 14 | 124427-124409 | AXACTCATCATCTGCGCTT |
| | 124462-124443 | TGGACGATGACATTCAGAXA |
| | 124491-124476 | TTGAACTCTGCTTAXA |
| BCR EX 19 | 145866-145849 | AXACGCGGTAGATGCCCA |
| | 145885-145871 | ATGTCCGTGGCCAXA |
| | 145891-145907 | GXAGGCTGCCTTCAGTG |

### Example 4

Detection of CpG Methylation Using Site-specific Reporter Probes with Bisulfite-modified Genomic DNA for Epigenetic Analysis of Imprinting Abnormalities and Tumor Specimens

The determination of cytosine methylation status of critical CpG dinucleotides is an area of emerging importance in clinical diagnostics. It is well-known that for certain genes, CpG methylation of key promoter and 5′ exon sites is associated with functional inactivation of the gene through transcriptional silencing. Furthermore, specific regions of mammalian genomes are subject to differential CpG methylation-mediated transcriptional inactivation based on the gender of the parent contributing the chromosome. “Gametic imprinting” is of clinical relevance particularly when such regions are prone to sporadic deletion or duplication events. In these cases, phenotypic effects vary with the parent-of-origin of the chromosomal region present in other than haploid number. Examples include the Prader-Willi/Angelman locus at 15q11-q13 and the Beckwith-Wiedemann locus at 11p15 (Hall J G. Genomic imprinting: nature and clinical relevance. Annu Rev Med 1997;48:35-44).

In the first case, deletions of the paternal chromosome are associated with the Prader-Willi syndrome (PWS) characterized by dysmorphisms, mental retardation, obesity and hypogonadism, while the identical deletion from the molecular standpoint occurring on the maternally inherited chromosome gives rise to the Angelman syndrome (AS) phenotype, comprising mental retardation with normal body habitus, ataxia, aphasia and seizures. PWS is believed to result from absence of transcripts expressed exclusively from the paternal chromosome while AS results from absence of maternally expressed transcripts. Further confounding molecular diagnostics is that both syndromes may result from parental isodisomy in which the normal complement of genetic material is present, but both chromosomes were contributed by the same parent with no contribution form the other. In this case, for instance, the PWS phenotype is observed in conjunction with maternal isodisomy for chromosome 15. Although all genes from the critical 15q11-q13 region are present in diploid copy number, expression patterns from both follow the maternal pattern (Hanel M L, Wevrick R. The role of genomic imprinting in human developmental disorders: lessons from Prader-Willi syndrome. Clin Genet 2000;59:156-64).

The Beckwith-Wiedemman syndrome (BWS) is characterized by neonatal overgrowth, often with hemihypertrophy, dysmorphisms, macroglossia, omphalocoele, hepatomegaly and a predisposition to renal and hepatic cancers. BWS is associated with duplications of genetic material from 11 p15 inherited from the father. For both the PWS/AS and BWS regions, characteristic CpG methylation sites have been identified that correlate with parent-of-origin specific expression patterns. Molecular diagnostics for these clinical entities entails a combination of assessment of chromosomal deletion or duplication, usually by fluorescent in situ hybridization (FISH), and analysis of CpG methylation status of defined residues, either by Southern blotting with methylation-sensitive restriction enzyme digestion or with bisulfite-modified PCR (American Society of Human Genetics/American College of Medical Genetics Test and Technology Transfer Committee. Diagnostic testing for Prader-Willi and Angelman syndromes: report of the ASHG/ACMG Test and Technology Transfer Committee. Am J Hum Genet 1996;58:1085-8). This latter approach entails treating genomic DNA with bisulfite that converts specifically unmethylated cytosine residues to uracil. PCR products obtained using these templates can be analyzed by restriction enzyme digestion, direct sequencing or HPLC (Herman J G, Graff J R, Myohanen S, Nelkin B D, Baylin S B. Methylation-specific PCR: a novel PCR assay for methylation status of CpG islands. Proc Natl Acad Sci USA 1996;93:9821-6).

A particularly important area of molecular diagnostics today surrounds the effects of CpG methylation mutations in cancer. Cancer is understood to arise from the sequential accumulation of mutations in tumor suppressor genes, oncogenes and genes whose products are of critical importance to apoptotic pathways or the cell cycle. Some of these mutations are point mutations, but the majority comprise abnormalities of gene copy number through chromosomal or segmental aneuploidies and abnormalities of CpG promoter methylation (Jones P A, Laird P W. Cancer epigenetics comes of age. Nat Genet 1999;21:163-7). Both of the latter exert their influence through altered transcription of critical genes. Genes for which CpG methylation abnormalities have been identified in tumor specimens include the following: ERα, RARbeta2, caspase 8, E-cadherin, P16INK4a/p14ARF, 14-3-3sigma, PR, BRCA1, GSTP1, FHIT, APC, p16, TMS1, hMLH1, VHL, RB1, p53, GSTP1, p73 and RASSF1. Analysis of aberrant CpG methylation from somatic tissues again relies mostly on bisulfite-modified PCR.

In certain cases, it is becoming clear that genetic and epigenetic (as processes involving functional modification of DNA without alteration of coding sequence are called) mutation analysis can enable parsing of clinically and histologically identical tumors into discrete subtypes with implications for prognosis and therapeutic response. One of the leading priorities in medical research today is the translation of these research findings into simple, accurate, robust and cost-effective tools to guide clinical care.

One of the difficulties inherent in assays for CpG methylation is that, in most cases, the CpG sites cluster in islands in promoter regions in which the majority—but rarely the totality—display a particular methylation pattern. Assays relying on detection of a single one or two CpG sites can be confounded by incomplete methylation or unmethylation, while sequencing-based methods that can look at multiple sites are costly and time-consuming. A better method would allow the simultaneous analysis of multiple sites at once with each site contributing a proportional degree of signal in an additive manner.

The reporter-specific method described above for homologous or paralogous sequences lends itself to this application. Here, a well-characterized region subject to differential CpG-methylation-dependent transcription is analyzed using a common capture probe and specific reporter probe sets on bisulfite-treated genomic DNA. Each of the reporter probes is designed to discriminate between the presence of C or U residues at defined sites through selective hybridization. The following assay for the PWS/AS SNRPN promoter and exon 1 region is proposed.

Determination of CpG Methylation Status in the Prader-Willi/Angelman Syndrome Imprinted Region of Chromosome 15q11-q13 by Oligonucleotide Hybridization with Reporter Probe-dependent Detection of Bisulfite Modification

A region of chromosome 15q11-q 13 was identified containing the SNRPN promoter and exon 1 sequence, including 23 well-defined CpG sites subject to parent-of-origin specific methylation (Zeschnigk M, Schmitz B, Dittrich B, Buiting K, Horsthemke B, Doerfier W. Imprinted segments in the human genome: different DNA methylation patterns in the Prader-Willi/Angelman syndrome region as determined by the genomic sequencing method. Hum Mol Genet 1997;6:387-95). A standard XLnt-based photocrosslinking assay as described above is proposed (Peoples R, Weltman H, Van Atta R, Wang J, Wood M, Ferrante-Raimondi M, Cheng P, et al. High-Throughput Detection of Submicroscopic Deletions and Methylation Status at 15q11-q 13 by a Photo-Cross-Linking Oligonucleotide Hybridization Assay. Clinical Chemistry 2002;48:1844-50). Reporter probes were designed as described above such that each probe sequence contained no fewer than 10% of variant C/U residues after bisulfite modification. A capture probe from an invariant region is included. “X” denotes the XLnt crosslinking nucleotide, as usual incorporated at either the 3′ or 5′ terminus (reporter probes) or both (capture probe). For the reporter probes, the designation “(G/A)” refers to the site specificity of the probe. One set of reporter probes will be generated with a G nucleotide at each of the designated positions (Probe set “G”). This G will hybridize specifically to target retaining the unmodified C, protected from bisulfite modification by the methylation of the residue. The alternate probe set will be generated with an A at each of the designated positions, specifically binding to modified sequences containing a U at the opposing residue (Probe set “A”). The bold-faced A denotes a position that would have opposed a necessarily unmethylated cytosine that is expected to undergo conversion to uracil. As described above, capture probes will be modified with biotin for reversible immobilization on magnetic beads; reporter probes will be polyfluoresceinated for signal elaboration. Nucleotide numbers correspond to GenBank accession number U41384.

| | | |
|---|---|---|
| REP | 15464-15445 | AXACAC(G/A)CCTAC(G/A)C |
| | | (G/A)ACC(G/A)C |
| REP | 15444-15426 | AXAAACAAACTAAC(G/A)C |
| | | (G/A)CA |
| REP | 15419-15401 | AAC(G/A)AAAATATATAC |
| | | (G/A)AXA |
| REP | 15387-15370 | CAAC(G/A)AATCTAAC(G/A) |
| | | CAXA |
| REP | 15368-15351 | TAAAAC(G/A)ACC(G/A)CC |
| | | (G/A)AAXA |
| REP | 15318-15300 | AAC(G/A)C(G/A)ATAAAAC |
| | | (G/A)AACXA |
| CAP | 15228-15199 | AXATTTTTAAAACTTAAAATAC |
| | | TAAATAXA |
| AlwI sites | 15155 and 15596 | |

The assay will be performed as described previously with the following modifications (Peoples R, Weltman H, Van Atta R, Wang J, Wood M, Ferrante-Raimondi M, Cheng P, et al. High-Throughput Detection of Submicroscopic Deletions and Methylation Status at 15q11-q13 by a Photo-Cross-Linking Oligonucleotide Hybridization Assay. Clinical Chemistry 2002;48:1844-50). Genomic DNA will be extracted from roughly 0.5 mls of anticoagulated blood for a minimum of 5 ugs; restriction digested with AlwI to generate 441 bp fragments containing target sequences; and treated overnight with bisulfite as described (Zeschnigk M, Schmitz B, Dittrich B, Buiting K, Horsthemke B, Doerfler W. Imprinted segments in the human genome: different DNA methylation patterns in the Prader-Willi/Angelman syndrome region as determined by the genomic sequencing method. Hum Mol Genet 1997;6:387-95). The sample will be denatured as described and aliquotted into each of two wells of a 96-well microtitre plate. To each well, hybridization solution and the common capture probe will be added. Each well will also receive either of the “G” or “A” reporter probe mix. Hybridization, photocrosslinking, high-stringency washing and signal elaboration will be performed as described (Peoples R, Weltman H, Van Atta R, Wang J, Wood M, Ferrante-Raimondi M, Cheng P, et al. High-Throughput Detection of Submicroscopic Deletions and Methylation Status at 15q11-q13 by a Photo-Cross-Linking Oligonucleotide Hybridization Assay. Clinical Chemistry 2002;48:1844-50). The signal obtained from each well will be corrected for background and a ratio obtained between the two with the “G” set represented by the numerator and the “A”, the denominator. It is anticipated that for the germline mutations of the PWS/AS region, ratios will fall into three discrete groups, clustering around 0.0, 1.0 and >10, corresponding to complete absence of methylation, normal hemimethylation, and complete methylation. As the crosslinking technology enables accurate gene dosage determination, it is possible to incorporate a third assay for an obligate dosage control in order to obtain a complete profile of the region. This assay must be designed taking into account the expected results of bisulfite modification.

Gene conversion mutations and paralogous loci

| | |
|---|---|
| CYP2D6 | Altered metabolism of 25% of common |
| | drugs |
| CYP2C9 | Risk of warfarin toxicity |
| CYP2C19 | Risk of toxicity with anticonvulsants |
| CYP21 | Congenital adrenal hyperplasia |
| α globin | α thallasemia |
| β globin | β thallasemia; hemglobinopathies |
| SMN1 | Spinal muscular atrophy |
| NCF1 | Chronic granulomatous disease |
| Rh locus | Immune-mediated hemolytic anemia |
| HLA locus | Xenograft (organ transplant) rejection |
| GH | Growth retardation |

Chromosomal inversions and translocations

| | |
|---|---|
| Ph chromosome | Chronic myelogenous leukemia |
| Factor VIII | Hemophilia A |

Imprinting disorders

| | |
|---|---|
| SNRPN | Prader-Willi/Angelman syndromes |
| H19 promoter | Beckwith-Wiedemann syndrome |
| HYMA1/ZAC | Transient Neonatal Diabetes Mellitus |

Somatic methylation mutations in cancer

| | |
|---|---|
| hMLH1 | Colorectal, gastric |
| P14ARF/p16INK4a | Colorectal, melanoma, ovarian, lung, etc. |
| VHL | Renal |
| RB1 | Retinoblastoma |
| p53 | Lung |
| E-cadherin | Esophageal |
| GSTP1 | Prostate |
| RARbeta2 | Prostate |
| FHIT | Lung, breast |
| p73 | Acute lymphoblastic leukemia |

All publications and patent applications mentioned in this specification are herein incorporated by reference to the same extent as if each individual publication or patent application was specifically and individually indicated to be incorporated by reference.

The invention now being fully described, it will be apparent to one of ordinary skill in the art that many changes and modifications can be made thereto without departing from the spirit or scope of the appended claims.

What is claimed is:

1. A method for genotyping a target nucleic acid sequence in a sample comprising sequences having high homology to the target sequence, wherein said target nucleic acid sequence comprises an interrogation region and a locus-specific region, said method comprising the steps of:
(a) adding a capture probe to said sample, wherein said capture probe is substantially complementary to at least a portion of said interrogation region of said target sequence;
(b) adding a reporter probe to said sample, wherein said reporter probe is substantially complementary to at least a portion of said locus-specific region of said target sequence
(c) capturing said capture probe; and
(d) detecting said reporter probe to determine the genotype of said target sequence and discriminate between said target sequence and said sequences having high homology to said target sequence.

2. The method according to claim 1, wherein said capture probe comprises a first label capable of being captured on a solid support.

3. The method according to claim 2, wherein said first label comprises biotin.

4. The method according to claim 1, wherein said reporter probe comprises a second label capable of providing a detectable signal.

5. The method according to claim 4, wherein said second label comprises a fluorophore.

6. The method according to any one of claims 1-5, wherein said capture and reporter probes further comprise a crosslinking agent, and said method further comprises an activating step prior to said capturing and detecting steps.

7. The method according to claim 6, wherein said crosslinking agent comprises a photoactivatable compound.

8. The method according to claim 7, wherein said photoactivatable compound comprises a coumarin derivative.

9. The method according to claim 7, wherein said photoactivatable compound comprises an aryl-olefin derivative.

10. The method according to any one of claims 6-9, wherein said method further comprises a high-stringency wash step after said activating step and prior to said capturing and detecting steps.

12. The method of claim 1, wherein said target sequence further comprises a dosage region and said method further comprises the addition and detection of a dosage probe having a sequence substantially complementary to at least a portion of said dosage region of said target sequence.
